(19) United States
(12) Patent Application Publication
(10) Pub. No.: US 2023/0420082 A1
(43) Pub. Date: Dec. 28, 2023

(54) GENERATING AND IMPLEMENTING A STRUCTURAL VARIATION GRAPH GENOME

(71) Applicant: Illumina Software, Inc., San Diego, CA (US)

(72) Inventors: Fan Zhang, San Diego, CA (US); Sean Truong, San Diego, CA (US)

(21) Appl. No.: 18/342,463

(22) Filed: Jun. 27, 2023

Related U.S. Application Data
(60) Provisional application No. 63/367,075, filed on Jun. 27, 2022.

(51) Int. Cl. G16B 45/00; G16B 30/00; G16B 20/00

(52) U.S. Cl. CPC G16B 45/00; G16B 30/00; G16B 20/00

(57) ABSTRACT

This disclosure describes methods, non-transitory computer readable media, and systems that can generate a structural variation graph genome with alternate contiguous sequences representing structural variant haplotypes. For instance, the disclosed systems can identify candidate structural variants that satisfy an occurrence threshold within a genomic sample database. From among the candidate structural variants, the systems select structural variant haplotypes based on one or both of the structural variant haplotypes satisfying a relative haplotype frequency and finding flanking variants adjacent to particular structural variant haplotypes. The systems can likewise select reference haplotypes corresponding to the selected structural variant haplotypes from a reference genome. Based on the selected haplotypes, the disclosed systems generate a structural variation graph genome comprising both alternate contiguous sequences representing the structural variant haplotypes and reference sequences representing the reference haplotypes.

### CROSS-REFERENCE TO RELATED APPLICATIONS

The present application claims the benefit of, and priority to, U.S. Provisional Application No. 63/367,075, entitled “GENERATING AND IMPLEMENTING A STRUCTURAL VARIATION GRAPH GENOME,” filed on Jun. 27, 2022. The aforementioned application is hereby incorporated by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor many thousands of oligonucleotides being synthesized in parallel from templates to predict nucleobase calls for growing nucleotide reads. A camera in many existing sequencing systems captures images of irradiated fluorescent tags incorporated into oligonucleotides. After capturing such images, some existing sequencing systems determine nucleobase calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing systems further utilize a variant caller to identify variants of a genomic sample, such as single nucleotide polymorphisms (SNPs), insertions or deletions (indels), or other variants.

Despite these recent advances, existing sequencing systems often generate or use reference genomes that misrepresent certain populations and foment inaccurate read alignment and mistaken variant calling. For example, some existing sequencing systems use a linear reference genome that purportedly represents a consensus or example of genes and other nucleotide sequences of an organism. But about 93% of the primary assembly for the most common linear human reference genome, GRCh38 from the Genome Reference Consortium, is based on libraries from only 11 individuals, with 70% of the linear human reference genome coming from 1 individual. Accordingly, existing systems use a linear human reference genome that often does not represent certain populations or common variants. Indeed, many linear human reference genomes fail to represent larger deletions or insertions (e.g., indels over 50 base pairs), translocations, inversions, copy number variations (CNVs), or other structural variants.

To address this lack of genetic representation in linear reference genomes, some existing sequencing systems generate or use a reference graph genome. For example, some reference graph genomes include both a linear reference genome and graph augmentations or alternate contiguous sequences that represent SNPs or small indels (e.g., 10 or fewer base pairs, 50 or fewer base pairs). While such reference graph genomes better represent some population's genetics, the expanded representation of existing reference graph genomes omits larger indels, translocations, inversions, or other structural variations that genomic samples frequently carry—similar to the shortcomings of existing linear reference genomes.

Because existing linear and graph reference genomes fail to represent structural variants, existing sequencing systems frequently misalign nucleotide reads of more diverse genomic samples with a reference genome and generate inaccurate variant or other nucleobase calls based on such misalignments. Indeed, in some cases, existing linear or graph reference genomes lack a graph augmentation or alternate contiguous sequence representing structural variants with which nucleotide reads can accurately align. Because existing reference genomes often fail to represent structural variants, existing sequencing systems also often fail to accurately determine when different segments of a nucleotide read best align with different portions of an existing reference genome in a split alignment. As a consequence of such split alignments or other complex alignments with structural variants, existing sequencing systems frequently generate incorrect variant calls that misidentify a presence or absence of a structural variant or provide no information on a relevant structural variant.

To compensate for the failure of some existing reference genomes to represent structural variants, some existing sequencing systems perform both whole genome sequencing (WGS) using an existing reference genome and SBS (or other techniques) and microarrays with genotyping probes that target specific structural variants. Indeed, microarrays have been specifically designed to target hard-to-detect structural variants using existing sequencing devices. By running both WGS and multiple microarrays—and sometimes using different specialized sequencing devices and microarray devices existing sequencing systems multiply the computer processing and time to determine accurate variant calls for both (i) SNPs and smaller indels and (ii) structural variants.

While some existing sequencing systems attempt to cure alignment-accuracy and base-calling-accuracy problems with graph reference genomes, existing graph reference genomes often include excessive augmentations for alleles similar enough (or irrelevant) to the alleles exhibited by many or a majority of genomic samples. For example, some existing sequencing systems utilize generic graph genomes that include large numbers of graph augmentations for alleles that are both common and uncommon across different populations, such as common and uncommon SNPs and small indels. Because such graph augmentations can be similar to—but not match—many sample genomes' alleles, generic graph genomes frequently cause existing sequencing systems to misalign or miss call variants for a large number of samples. By utilizing generic graph reference genomes with excessive graph augmentations, therefore, existing sequencing systems can increase the chances of mismatched alignments with nucleotide reads from a genomic sample.

In addition to alignment-accuracy and nucleobase-accuracy problems, some existing graph reference genomes are bulky and consume considerable memory and computing resources. Indeed, some existing graph reference genomes can include countless graph augmentations for SNPs or small indels that are irrelevant to a given genomic sample. These countless alternative paths can consume unnecessary memory. In addition to wasting memory, generic graph reference genomes often increase the computer processing time for existing sequencing systems to determine whether to include or exclude matches to graph augmentations when making variant calls.

These, along with additional problems and issues exist in existing sequencing systems.

### SUMMARY

This disclosure describes one or more embodiments of systems, methods, and non-transitory computer readable storage media that solve one or more of the problems described above or provide other advantages over the art. In particular, the disclosed system can generate or implement a structural variation graph genome with alternate contiguous sequences representing structural variant haplotypes. For instance, the disclosed systems can identify candidate structural variants that satisfy an occurrence threshold within a genomic sample database. From among the candidate structural variants, the systems select structural variant haplotypes based on one or both of the structural variant haplotypes satisfying a relative haplotype frequency and finding flanking variants adjacent to particular structural variant haplotypes. The systems can likewise select reference haplotypes corresponding to the selected structural variant haplotypes from a reference genome. Based on the selected haplotypes, the systems generate a structural variation graph genome comprising both alternate contiguous sequences representing the structural variant haplotypes and reference sequences representing the reference haplotypes. Based on comparing nucleotide reads of a genomic sample with alternate contiguous sequences representing structural variant haplotypes, the disclosed systems can determine nucleobase calls (e.g., structural variant calls) for the genomic sample.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which a structural-variant-aware sequencing system can operate in accordance with one or more embodiments of the present disclosure.

FIG. 2A illustrates a schematic diagram of the structural-variant-aware sequencing system generating a structural variation graph genome comprising alternate contiguous sequences representing structural variant haplotypes and reference sequences representing reference haplotypes in accordance with one or more embodiments of the present disclosure.

FIG. 2B illustrates a schematic diagram of the structural-variant-aware sequencing system aligning nucleotide reads of a genomic sample with a structural variation graph genome and determining nucleobase calls for the genomic sample based on the aligned nucleotide reads in accordance with one or more embodiments of the present disclosure.

FIG. 3 illustrates a schematic diagram of the structural-variant-aware sequencing system selecting structural variant haplotypes for a target genomic region of a structural variation graph genome based on one or both of a phasing criteria and a region occurrence threshold in accordance with one or more embodiments of the present disclosure.

FIG. 4 illustrates the structural-variant-aware sequencing system combining reference sequences from a reference genome, selected structural variant haplotypes, and selected alternate haplotypes into a structural variation graph genome using a hash table in accordance with one or more embodiments of the present disclosure.

FIG. 5 illustrates the structural-variant-aware sequencing system aligning nucleotide reads of a genomic sample with a structural variation graph genome and determining nucleobase calls for the genomic sample based on the aligned nucleotide reads in accordance with one or more embodiments of the present disclosure.

FIG. 6 illustrates a client device displaying a graphical user interface comprising variant calls for structural variant haplotypes in accordance with one or more embodiments of the present disclosure.

FIG. 7 illustrates a table that shows different accuracy measurements of (i) a sequencing system determining variant calls for deletions and insertions exceeding 50 base pairs using an existing graph reference genome that lacks alternate contiguous sequences representing structural variants and (ii) the structural-variant-aware sequencing system determining structural variant calls for such deletions and insertions using a structural variation graph genome in accordance with one or more embodiments of the present disclosure.

FIG. 8 illustrate series of acts for generating a structural variation graph genome in accordance with one or more embodiments of the present disclosure.

FIG. 9 illustrate series of acts for aligning nucleotide reads of a genomic sample with a structural variation graph genome and determining nucleobase calls for the genomic sample based on the aligned nucleotide reads in accordance with one or more embodiments of the present disclosure.

FIG. 10 illustrates a block diagram of an example computing device in accordance with one or more embodiments of the present disclosure.

### DETAILED DESCRIPTION

This disclosure describes one or more embodiments of a structural-variant-aware sequencing system that can generate a structural variation graph genome with alternate contiguous sequences representing structural variant haplotypes selected from candidate structural variants. For instance, the structural-variant-aware sequencing system can identify candidate structural variants of a threshold frequency (or that otherwise satisfy another occurrence threshold) within a genomic sample database. Such candidate structural variants may include a deletion or insertion exceeding a threshold number of base pairs (e.g., 50), a duplication, an inversion, a translocation, a copy number variation (CNV), or other structural variant. From among the candidate structural variants, the structural-variant-aware sequencing system selects structural variant haplotypes based on one or both of satisfying another occurrence threshold and finding flanking variants adjacent to particular structural variant haplotypes. The system can likewise select reference haplotypes of genomic regions corresponding to the selected structural variant haplotypes from a reference genome. Based on the selected haplotypes, the system generates a structural variation graph genome comprising both alternate contiguous sequences representing the structural variant haplotypes and reference sequences representing the reference haplotypes.

As suggested above, the structural-variant-aware sequencing system can identify candidate structural variants from a genomic sample database based on an occurrence threshold. For instance, the structural-variant-aware sequencing system can identify candidate structural variants that satisfy a particular variant frequency or a minimum count in a genomic sample database. Such a genomic sample database may include a digital catalogue of nucleotide reads, whole genomes, exomes, exons, or other nucleotide sequences from a diverse set of genomic samples. When identifying candidate structural variants, the structural-variant-aware sequencing system may identify deletions or insertions exceeding a threshold number of base pairs (e.g., >50 base pairs) or various other structural variants at various genomic regions across a linear reference genome. From within the genomic sample database, the structural-variant-aware sequencing system can identify such candidate structural variants from long nucleotide reads or other contiguous sequences.

From among the identified candidate structural variants, in certain implementations, the structural-variant-aware sequencing system selects structural variant haplotypes. For instance, in some cases, the structural-variant-aware sequencing system selects structural variant haplotypes that satisfy a threshold frequency or a threshold count at target genomic regions corresponding to the candidate structural variants. Additionally or alternatively, the structural-variant-aware sequencing system selects structural variant haplotypes that are in phase with flanking variants within contiguous sequences of the genomic sample database. Such flanking variants may include SNPs or indels of less than a threshold number of base pairs (e.g., <50 base pairs).

After selecting structural variant haplotypes, in some embodiments, the structural-variant-aware sequencing system integrates the structural variant haplotypes and reference haplotypes from a linear reference genome into a data organization structure. For instance, in certain implementations, the structural-variant-aware sequencing system maps reference haplotypes from the linear reference genome, SNPs, structural variant haplotypes to genomic coordinates within the linear reference genome. The structural-variant-aware sequencing system can further associate nucleobase identifiers (e.g., letters for A, T, C, G, U) for the mapped reference haplotypes, SNPs, and structural variant haplotypes with values representing the genomic coordinates in an organizational structure (e.g., hash table, matrix).

In addition or in the alternative to generating a structural variation graph genome, in some embodiments, the structural-variant-aware sequencing system determines nucleobase calls for a genomic sample based on comparing nucleotide reads of the genomic sample with the structural variation graph genome. For instance, in some embodiments, the structural-variant-aware sequencing system identifies nucleotide reads from a genomic sample. The structural-variant-aware sequencing system further aligns a subset of nucleotide reads with an alternate contiguous sequence representing a structure variant haplotype within a structural variation graph genome. Based on the aligned subset of nucleotide reads, the structural-variant-aware sequencing system generates nucleobase calls (e.g., variant calls) for the genomic sample.

In addition to generating nucleobase calls, in some embodiments, the structural-variant-aware sequencing system reports various data corresponding to the nucleobase calls corresponding to a structural variant haplotype. For instance, in some cases, the structural-variant-aware sequencing system generates an alignment file or a variant call file comprising an annotation indicating a structural variant haplotype, a frequency of the structural variant haplotype, or genomic coordinates for the structural variant haplotype corresponding to the nucleobase calls.

Beyond reporting variant calls corresponding to structural variants, the structural-variant-aware sequencing system can better align and generate variant calls for split-read alignments. As suggested above, the structural-variant-aware sequencing system can determine when nucleotide reads align with structural variant haplotypes. For example, in certain cases, the structural-variant-aware sequencing system determines that a subset of nucleotide reads overlap with a breakpoint of an alternate contiguous sequence representing a structural variant haplotype in the structural variation graph genome. Based on detecting such overlap, the structural-variant-aware sequencing system generates an alignment file or a variant call file with an annotation indicating an alignment reflecting the structural variant haplotype within the genomic sample.

As indicated above, the structural-variant-aware sequencing system provides several technical advantages relative to existing sequencing systems by improving read-alignment and base-calling accuracy, computational efficiency, and memory consumption relative to existing sequencing systems. For example, the structural-variant-aware sequencing system improves the accuracy of read alignments and nucleobase calling by generating or utilizing a structural variation graph genome that accounts for structural variants. Unlike existing linear reference genomes or existing graph reference genomes that fail to accurately or adequately represent structural variants, the structural-variant-aware sequencing system can generate or implement a structural variation graph genome comprising alternate contiguous sequences representing structural variant haplotypes. By selecting structural variant haplotypes that are respectively in phase with flanking variants within contiguous sequences, in some cases, the structural-variant-aware sequencing system incorporates structural variant haplotypes into alternate contiguous sequences that facilitate better alignment between (i) nucleotide reads reflecting such flanking variants and structural variants and (ii) intelligently selected alternate contiguous sequences of the structural variation graph genome. By further or alternatively selecting structural variant haplotypes that satisfy occurrence thresholds at targeted genomic regions, in some cases, the structural-variant-aware sequencing system incorporates structural variant haplotypes into alternate contiguous sequences that efficiently facilitate better alignment between nucleotide reads reflecting more common structural variant haplotypes and the selected alternate contiguous sequences of the structural variation graph genome.

Regardless of the disclosed selection approach, the structural-variant-aware sequencing system's alternate contiguous sequences facilitate improved alignment with nucleotide reads indicating larger indels, translocations, inversions, CNVs, or other structural variants. Because of the improved alignment with the structural variation graph genome, the structural-variant-aware sequencing system can also determine more accurate nucleobase calls with a higher confidence that such calls match (or differ from) the reference bases of a reference genome than existing sequencing systems. Indeed, the disclosed structural variation graph genome facilitates variant calls or other nucleobase calls that existing reference genomes do not (or cannot) facilitate with a same quality (e.g., Q score) or mapping quality (e.g., MAPQ).

In addition to improving alignment and base-calling accuracy, the structural-variant-aware sequencing system improves the computing speed and memory of some sequencing systems using graph reference genomes. In contrast to a generic graph reference genome that would include graph augmentations for irrelevant or excessive alleles—and that indiscriminately represent countless SNPs and/or small indels (e.g., 10 or fewer base pairs)—the structural-variant-aware sequencing system reduces the memory required to save a relatively smaller structural variation graph genome than a genic graph reference genome of countless graph augmentations. Rather than inefficiently using computing resources, such as processing and memory storage, on deciding between an excessive number of possible read-alignment matches with indiscriminate alternate contiguous sequences for SNPs, small indels, or structural variants in a hypothetical generic graph reference genome, the structural-variant-aware sequencing system conserves computer processing and other resources by using a structural variation graph genome. To conserve such computing resources, in some embodiments, the structural variation graph genome comprises (i) fewer (but more relevant) alternate contiguous sequences representing selected flanking variants and corresponding structural variant haplotypes with which to compare a sample's genomic regions and (ii) more efficient mapping due to fewer candidate alternate-contiguous-sequence matches than a hypothetical generic graph reference genome comprising an indiscriminate number of alternate contiguous sequences comprising SNPs, small indels, or structural variants.

Beyond the improved computing efficiency of a structural variation graph genome with targeted alternate contiguous sequences, in some embodiments, the structural-variant-aware sequencing system improves computational efficiency by reducing the number of sequencing assays and computational devices used to determine variant calls for structural variants. As noted above, some existing sequencing systems consume significant computer processing and time by running both (i) WGS on a specialized sequencing device to generate nucleotide reads for a genomic sample and (ii) multiple genotyping microarrays on a microarray device. By comparing the nucleotide reads to a reference genome for WGS and analyzing light signals from DNA probes in a microarray, existing sequencing systems can determine accurate variant calls for both SNPs and smaller indels based on a reference genome, on the one hand, and targeted structural variants from DNA probes, on the other hand. In contrast to such existing sequencing systems, in some embodiments, the structural-variant-aware sequencing system facilitates a more computationally efficient approach by using a specialized sequencing device to determine nucleotide reads—without or with fewer genotyping microarrays for targeted structural variants—to determine variant calls corresponding to structural variants. Accordingly, the structural-variant-aware sequencing system can obviate some or all genotyping microarrays for structural variants by generating or utilizing a structural variation graph genome with alternate contiguous sequences representing structural variant haplotypes.

As illustrated by the foregoing discussion, the present disclosure utilizes a variety of terms to describe features and advantages of the structural-variant-aware sequencing system. As used herein, for example, the term “structural variant” refers to a variation (e.g., deletion, insertion, translocation, inversion) in a structure of an organism's chromosome or a variation to the nucleotide sequences of the organism's chromosome. In some cases, a structural variant includes a variation to a threshold number of base pairs (e.g., >50 base pairs) within an organism's chromosome. Accordingly, in certain implementations, a structural variant includes an insertion or deletion exceeding a threshold number of base pairs, a duplication exceeding a threshold number of base pairs, an inversion, a translocation, or a copy number variation (CNV). While this disclosure describes some examples of 50 base pairs as a threshold number of base pairs, in some embodiments, the threshold number of base pairs for a structural variant may be different, such as 35, 45, 100, or 1,000 base pairs.

Relatedly, the term “candidate structural variant” refers to a structural variant selected from a genomic sample database. In some cases, a candidate structural variant includes a structural variant that satisfies a threshold quantity of occurrences within a genomic sample database. For example, a candidate structural variant can include a structural variant from a genomic sample database that satisfies a threshold frequency or a threshold count at a target genomic region (e.g., a gene or promoter region) for the nucleotide sequences within the genomic sample database.

As just indicated, the structural-variant-aware sequencing system can select candidate structural variants from a genomic sample database. As used herein, the term “genomic sample database” refers to a database of digitally represented nucleotide sequences from genomic samples that comprises an organization, index, or search function to identify variants, reference alleles, or reference haplotypes. For instance, a genomic sample database can include (i) digitally represented nucleotide reads, whole genomes, exomes, exons, or other nucleotide sequences from a diverse set of genomic samples and (ii) an organization or index for genomic coordinates or regions by which digitally represented nucleotide sequences for variants or reference allele or haplotypes can be identified. To illustrate, in some embodiments, a genomic sample database includes one or more of the International Genome Sample Resource (IGSR) from the 1000 Genomes Project, the Genome Aggregation Database (gnomAD), the Database of Genomic Variants (DGV), or other databases that include nucleotide sequences representing structural variants, such as databases comprising nucleotide reads over 300 base pairs. In some cases, a genomic sample database represents a subset of nucleotide sequences selected from one or more of the aforementioned databases or other databases.

As noted above, in some embodiments, the structural-variant-aware sequencing system selects structural variant haplotypes from among candidate structural variants within a genomic sample database. As used herein, the term “structural variant haplotype” refers to a structural variant that is present in an organism (or organisms from a population) and that is inherited from one or more ancestors as part of a grouping of nucleotide sequences. In particular, a structural variant haplotype can include a group of alleles including (or representing) one or more structural variants present in organisms of a population that tend to be inherited together by such organisms from a single parent. Accordingly, a structural variant haplotype may include a structural variant and other variants as part of a group of alleles and may correspond to a particular gene.

By contrast, the term “reference haplotype” refers to a group of nucleotide sequences represented by a reference genome that is inherited from one or more ancestors as part of a grouping of a nucleotide sequence. In particular, a reference haplotype can include a group of alleles from a linear reference genome that tends to be inherited together by such organisms from a single parent. In some cases, a reference haplotype includes a group of alleles corresponding to a gene.

As also used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. While GRCh38 may include alternate contiguous sequences representing alternate haplotypes, such as SNPs and small indels (e.g., 10 or fewer base pairs, 50 or fewer base pairs), GRCh38 includes alternate haplotypes with limited representation of population structural variants. Indeed, the structural variants represented in GRCh38 include only those represented by the 11 individuals whose libraries GRCh38 is constructed upon.

Additionally, as used herein, the term “graph reference genome” refers to a reference genome that includes both a linear reference genome and alternate contiguous sequences (or graph augmentations) representing variant haplotype sequences or other variant or alternative nucleic-acid sequences. For instance, a graph reference genome can include a linear reference genome and alternate contiguous sequences corresponding to one or more population haplotype sequences identified from a genomic sample database. As an example, a graph reference genome may include the Illumina DRAGEN Graph Reference Genome hg19.

As disclosed herein, the term “structural variation graph genome” refers to a graph reference genome that includes alternate contiguous sequences representing structural variant haplotypes and reference sequences representing reference haplotypes. For instance, in some embodiments, a structural variation graph genome includes a linear reference genome that has been supplemented with alternate contiguous sequences representing structural variant haplotypes. In addition to such alternate contiguous sequences, in some embodiments, a structural variation graph genome comprises alternate nucleobases or additional alternate contiguous sequences representing alternate haplotypes, such as SNPs and/or indels below a threshold number of base pairs (e.g., <50 base pairs). While this disclosure uses the term structural variation graph genome, the structural-variant-aware sequencing system can represent and use the structural variation graph genome in the form of a graph hash table or other digital organization structure.

As further used herein, the term “contiguous sequence” (or simply “contig”) refers to a consensus nucleotide sequence for a genomic region of a genomic sample (or multiple genomic samples of a species) based on a set of overlapping nucleotide segments corresponding to the genomic region. In particular, a contiguous sequence includes a consensus nucleotide sequence for a genomic region of one or more genomic samples based on nucleotide reads for the one or more genomic samples covering (or overlapping with) the genomic region.

Relatedly, the term “alternate contiguous sequence” (or simply “alt contig”) refers to a contiguous sequence representing a population haplotype added to a linear reference genome (or other reference genome) at a particular genomic coordinate or genomic coordinates (e.g., lifted over to the linear reference genome). In some implementations, a structural variation graph genome can include alternate contiguous sequences mapped to genomic coordinates of a primary assembly for a linear reference genome. For example, an alternate contiguous sequence may represent a population haplotype containing a structural variant with liftover to two or more genomic coordinates in the linear reference genome corresponding to two or more flanks of structural variant breakends. In some cases, a hash table for a structural variation graph genome includes identifiers that associate alternate contiguous sequences representing structural variant haplotypes with genomic coordinates representing reference haplotypes from a primary assembly for a linear reference genome.

Additionally, as used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide-base within the source for the reference genome (e.g., mt:16568 or SARS-CoV-2:29001). By contrast, in certain cases, a genomic coordinate refers to a position of a nucleotide-base within a reference genome without reference to a chromosome or source (e.g., 29727).

As used herein, a “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1:1234570-1234870). In various implementations, a genomic coordinate includes a position within a reference genome. In some cases, a genomic coordinate is specific to a particular reference genome.

As further used herein, the term “reference sequence” refers to a nucleotide sequence from a reference genome. For instance, a reference sequence includes a sequence of nucleobases digitally represented by a primary assembly of a linear reference genome. As suggested above, in some embodiments, a reference sequence digitally represents a reference haplotype from the primary assembly of the linear reference genome.

As further used herein, the term “flanking variant” refers to a variant nucleobase or multiple variant nucleobases that do not align with or differ from a corresponding nucleobase or nucleobases of a reference genome and that is adjacent to (or part of) a structural variant haplotype within a nucleotide sequence. For example, a flanking variant includes a variant nucleobase or multiple variant nucleobases that do not align with or differ from a reference nucleobase or reference nucleobases and that are in phase with a structural variant haplotype within a nucleotide sequence (e.g., contiguous sequence) from a genomic sample database. As suggested above, a flanking variant may include an SNP, a deletion of less than a threshold number of base pairs, or an insertion of less than the threshold number of base pairs. In some cases, a flanking variant may also be a structural variant.

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or another base-call-output file—based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or a uracil (U) call.

As further used herein, the term “nucleotide read” (or simply “read”) refers to an inferred sequence of one or more nucleobases (or nucleobase pairs) from all or part of a sample nucleotide sequence (e.g., a sample genomic sequence, cDNA). In particular, a nucleotide read includes a determined or predicted sequence of nucleobase calls for a nucleotide sequence (or group of monoclonal nucleotide sequences) from a sample library fragment corresponding to a genome sample. For example, in some cases, a sequencing device determines a nucleotide read by generating nucleobase calls for nucleobases passed through a nanopore of a nucleotide-sample slide, determined via fluorescent tagging, or determined from a cluster in a flow cell.

As further used herein, the term “alignment score” refers to a numeric score, metric, or other quantitative measurement evaluating an accuracy of an alignment between a nucleotide read or a fragment of the nucleotide read and another nucleotide sequence from a reference genome. In particular, an alignment score includes a metric indicating a degree to which the nucleobases of a nucleotide read match or are similar to a reference sequence or an alternate contiguous sequence from a reference genome. In certain implementations, an alignment score takes the form of a Smith-Waterman score or a variation or version of a Smith-Waterman score for local alignment, such as various settings or configurations used by DRAGEN by Illumina, Inc. for Smith-Waterman scoring.

Relatedly, the term “alt-contig fragment alignment score” refers to an alignment score for an alignment between one or more read fragments with an alternate contiguous sequence. In particular, an alt-contig fragment alignment score can include an alignment score for an alignment of one or more inner read fragments and one or more outer read fragments of a nucleotide read with an alternate contiguous sequence. As explained below, an alt-contig fragment alignment score may replace or serve as a split group score under certain circumstances.

As further used herein, the term “alignment file” refers to a digital file that indicates the relative alignment or mapping of nucleotide reads with nucleotide sequences of a reference genome or other reference nucleotide sequences. In particular, an alignment file can include data indicating relative mapping position of nucleotide reads and nucleotide sequences of a reference genome. In some embodiments, an alignment file includes or constitutes a Sequence Alignment/Map (SAM) file, a Binary Alignment Map (BAM) file, a FAST-All (FASTA) file, or a FASTQ file.

As used herein, for example, the term “configurable processor” refers to a circuit or chip that can be configured or customized to perform a specific application. For instance, a configurable processor includes an integrated circuit chip that is designed to be configured or customized on site by an end user's computing device to perform a specific application. Configurable processors include, but are not limited to, an ASIC, ASSP, a coarse-grained reconfigurable array (CGRA), or FPGA. By contrast, configurable processors do not include a CPU or GPU. In some embodiments, the structural-variant-aware sequencing system uses a configurable processor (e.g., FPGA) or a processor (e.g., CPU) to perform the various embodiments described herein.

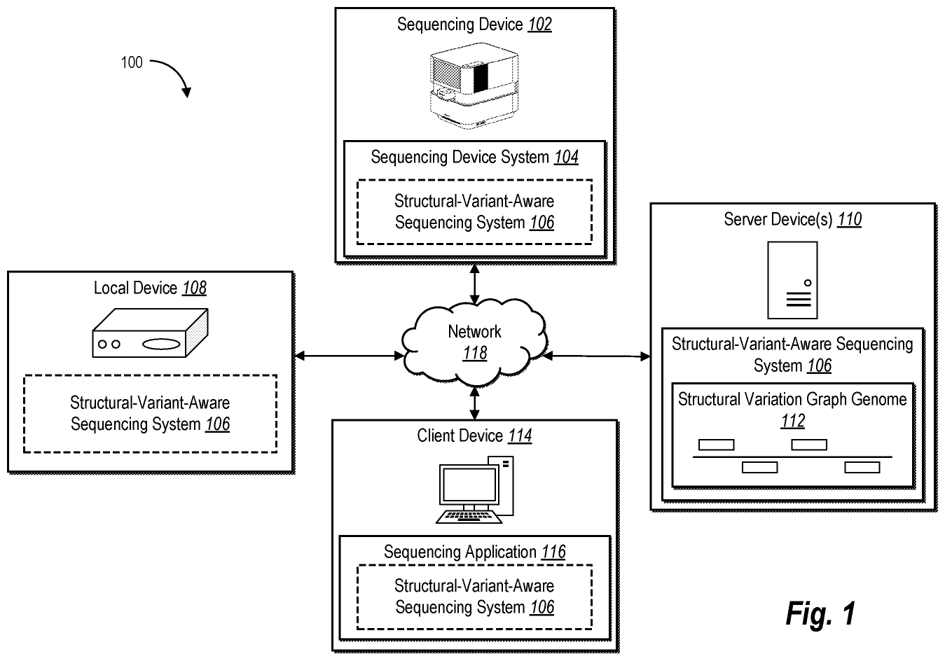

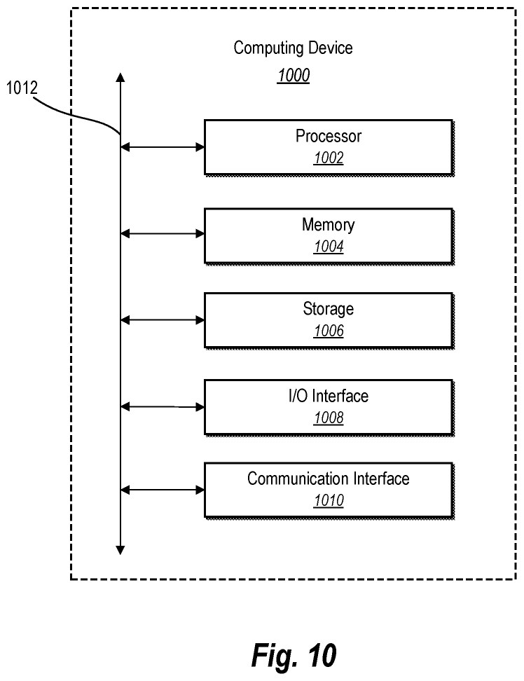

The following paragraphs describe the structural-variant-aware sequencing system with respect to illustrative figures that portray example embodiments and implementations. For example, FIG. 1 illustrates a schematic diagram of a computing system 100 in which a structural-variant-aware sequencing system 106 operates in accordance with one or more embodiments. As illustrated, the computing system 100 includes a sequencing device 102 connected to a local device 108 (e.g., a local server device), one or more server device(s) 110, and a client device 114. As shown in FIG. 1, the sequencing device 102, the local device 108, the server device(s) 110, and the client device 114 can communicate with each other via a network 118. The network 118 comprises any suitable network over which computing devices can communicate. Example networks are discussed in additional detail below with respect to FIG. 10. While FIG. 1 shows an embodiment of the structural-variant-aware sequencing system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments extracted from samples and further copies and determines the nucleobase sequence of such extracted nucleotide fragments.

In one or more embodiments, the sequencing device 102 utilizes SBS to sequence nucleotide fragments into nucleotide reads and determine nucleobase calls for the nucleotide reads. In addition or in the alternative to communicating across the network 118, in some embodiments, the sequencing device 102 bypasses the network 118 and communicates directly with the local device 108 or the client device 114. By executing the sequencing device system 104, the sequencing device 102 can further store the nucleobase calls as part of base-call data that is formatted as a binary base call (BCL) file and send the BCL file to the local device 108 and/or the server device(s) 110.

As further indicated by FIG. 1, the local device 108 is located at or near a same physical location of the sequencing device 102. Indeed, in some embodiments, the local device 108 and the sequencing device 102 are integrated into a same computing device. The local device 108 may run the structural-variant-aware sequencing system 106 to generate, receive, analyze, store, and transmit digital data, such as by receiving base-call data or determining variant calls based on analyzing such base-call data. As shown in FIG. 1, the sequencing device 102 may send (and the local device 108 may receive) base-call data generated during a sequencing run of the sequencing device 102. By executing software in the form of the structural-variant-aware sequencing system 106, the local device 108 may align nucleotide reads with a structural variation graph genome 112 and determine genetic variants based on the aligned nucleotide reads. The local device 108 may also communicate with the client device 114. In particular, the local device 108 can send data to the client device 114, including a variant call file (VCF) or other information indicating nucleobase calls, sequencing metrics, error data, or other metrics.

As further indicated by FIG. 1, the server device(s) 110 are located remotely from the local device 108 and the sequencing device 102. Similar to the local device 108, in some embodiments, the server device(s) 110 include a version of the structural-variant-aware sequencing system 106. Accordingly, the server device(s) 110 may generate, receive, analyze, store, and transmit digital data, such as by receiving base-call data or determining variant calls based on analyzing such base-call data. As indicated above, the sequencing device 102 may send (and the server device(s) 110 may receive) base-call data from the sequencing device 102. The server device(s) 110 may also communicate with the client device 114. In particular, the server device(s) 110 can send data to the client device 114, including VCFs or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As indicated above, as part of the server device(s) 110 or the local device 108, the structural-variant-aware sequencing system 106 can generate or implement a structural variation graph genome with alternate contiguous sequences representing structural variant haplotypes. For instance, the structural-variant-aware sequencing system 106 can identify candidate structural variants of a threshold frequency (or that otherwise satisfy another occurrence threshold) within a genomic sample database. From among the candidate structural variants, the structural-variant-aware sequencing system 106 selects structural variant haplotypes based on one or both of satisfying another occurrence threshold and finding flanking variants adjacent to particular structural variant haplotypes. The structural-variant-aware sequencing system 106 can likewise select reference haplotypes of genomic regions corresponding to the selected structural variant haplotypes from a reference genome. Based on the selected haplotypes, the structural-variant-aware sequencing system 106 generates a structural variation graph genome comprising both alternate contiguous sequences representing the structural variant haplotypes and reference sequences representing the reference haplotypes. Based on comparing nucleotide reads of a genomic sample with alternate contiguous sequences representing structural variant haplotypes, the structural-variant-aware sequencing system 106 can determine nucleobase calls for the genomic sample.

As further illustrated and indicated in FIG. 1, by executing a sequencing application 116, the client device 114 can generate, store, receive, and send digital data. In particular, the client device 114 can receive sequencing data from the local device 108 or receive call files (e.g., BCL) and sequencing metrics from the sequencing device 102. Furthermore, the client device 114 may communicate with the local device 108 or the server device(s) 110 to receive a VCF comprising nucleobase calls and/or other metrics, such as a base-call-quality metrics or pass-filter metrics. The client device 114 can accordingly present or display information pertaining to variant calls or other nucleobase calls within a graphical user interface of the sequencing application 116 to a user associated with the client device 114. For example, the client device 114 can present structural variant calls and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 10.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the structural-variant-aware sequencing system 106 and present, for display at the client device 114, base-call data or data from a VCF. Furthermore, the sequencing application 116 can instruct the client device 114 to display summaries for multiple sequencing runs.

As further illustrated in FIG. 1, a version of the structural-variant-aware sequencing system 106 may be located and implemented (e.g., entirely or in part) on the client device 114 or the sequencing device 102. In yet other embodiments, the structural-variant-aware sequencing system 106 is implemented by one or more other components of the computing system 100, such as the local device 108. In particular, the structural-variant-aware sequencing system 106 can be implemented in a variety of different ways across the sequencing device 102, the local device 108, the server device(s) 110, and the client device 114. For example, the structural-variant-aware sequencing system 106 can be downloaded from the server device(s) 110 to the structural-variant-aware sequencing system 106 and/or the local device 108 where all or part of the functionality of the structural-variant-aware sequencing system 106 is performed at each respective device within the computing system 100.

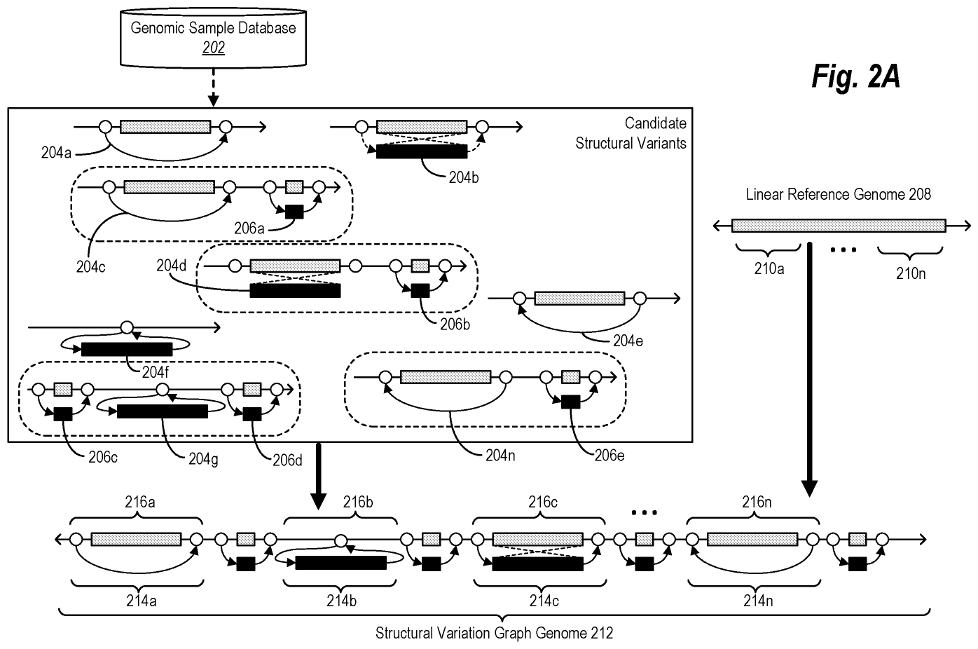

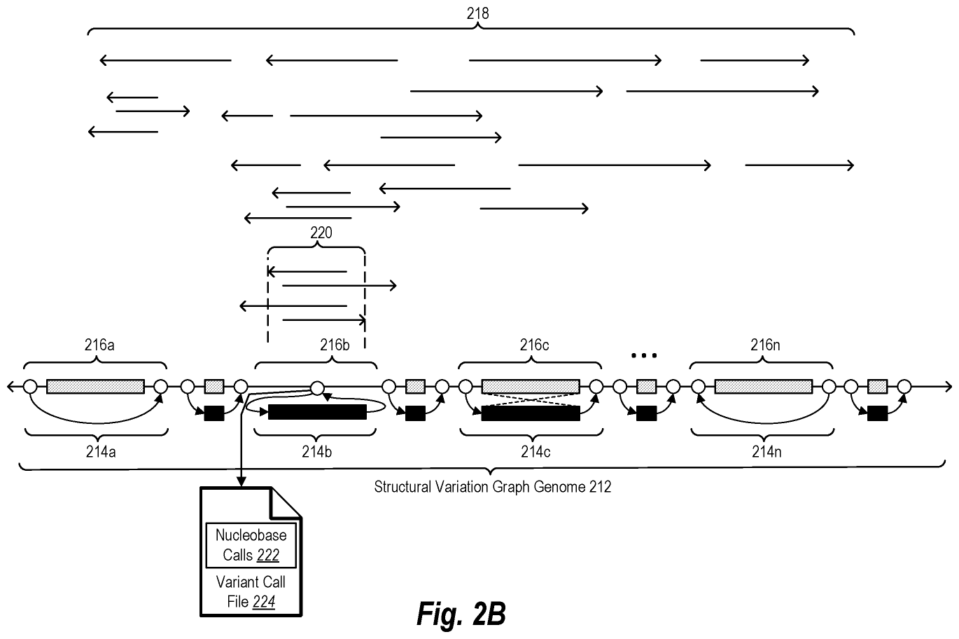

As indicated above, the structural-variant-aware sequencing system 106 can generate and implement a structural variation graph genome. FIGS. 2A and 2B depict an overview of such embodiments for the structural-variant-aware sequencing system 106. In accordance with one or more embodiments, FIG. 2A illustrates an example of the structural-variant-aware sequencing system 106 generating a structural variation graph genome 212 comprising alternate contiguous sequences representing structural variant haplotypes and reference sequences representing reference haplotypes. In accordance with one or more embodiments, FIG. 2B illustrates an example of the structural-variant-aware sequencing system 106 aligning nucleotide reads of a genomic sample with the structural variation graph genome 212 and determining nucleobase calls for the genomic sample based on the aligned nucleotide reads.

As shown in FIG. 2A, the structural-variant-aware sequencing system 106 identifies candidate structural variants 204a-204n from a genomic sample database 202 based on an occurrence threshold. For example, the structural-variant-aware sequencing system 106 identifies the candidate structural variants 204a-204n that satisfy a threshold quantity of occurrences within the genomic sample database 202. By selecting structural variants that satisfy a threshold count (e.g., 3 or more occurrences) or that satisfy a threshold frequency (e.g., 10%, 30% variant frequency) at target genomic coordinates in the genomic sample database 202, in some implementations, the structural-variant-aware sequencing system 106 selects the candidate structural variants 204a-204n from the genomic sample database 202. As noted above, the genomic sample database 202 may include a variety of databases comprising nucleotide reads from a diverse set of genomic samples, such as a combination of one or more of the IGSR from the 1000 Genomes Project, gnomAD, or the DGV.

As further indicated by FIG. 2A, the structural-variant-aware sequencing system 106 identifies a variety of structural-variant types among the candidate structural variants 204a-204n. Based on satisfying a threshold quantity of occurrence, for instance, the structural-variant-aware sequencing system 106 identifies the candidate structural variants 204a and 204c exhibiting deletions exceeding a threshold number of base pairs; the candidate structural variants 204b and 204d exhibiting translocations; the candidate structural variants 204f and 204g exhibiting insertions exceeding a threshold number of base pairs; and the candidate structural variants 204e and 204n exhibiting duplications exceeding a threshold number of base pairs. For illustrative purposes and space constraints, FIG. 2A depicts the candidate structural variants 204a-204n as merely examples. The structural-variant-aware sequencing system 106 may identify, from the genomic sample database 202, different types of structural variants (e.g., translocations, CNVs) and additional structural variants not depicted in FIG. 2A.

From among the candidate structural variants 204a-204n, as further shown in FIG. 2A, the structural-variant-aware sequencing system 106 selects structural variant haplotypes. In some cases, the structural-variant-aware sequencing system 106 selects structural variant haplotypes that satisfy an additional threshold quantity of occurrences at particular genomic regions, as categorized in the genomic sample database 202. For example, in certain implementations, the structural-variant-aware sequencing system 106 selects structural variant haplotypes that satisfy a threshold variant frequency (e.g., 15%, 25%) or a threshold count (3, 10) at target genomic coordinates corresponding to the candidate structural variants 204a-204n.

In addition or in the alternative to an occurrence threshold, in some embodiments, the structural-variant-aware sequencing system 106 selects structural variant haplotypes that are adjacent to flanking variants within contiguous sequences of the genomic sample database 202. In some cases, the flanking variants are in phase with respective structural variant haplotypes in nucleotide sequences of the genomic sample database 202. As indicated by FIG. 2A, for instance, the structural-variant-aware sequencing system 106 determines the candidate structural variant 204c is in phase with a flanking variant 206a within a contiguous sequence (or other nucleotide sequence) of the genomic sample database 202. Similarly, the structural-variant-aware sequencing system 106 determines the candidate structural variant 204d is in phase with a flanking variant 206b, the candidate structural variant 204g is in phase with flanking variants 206c and 206d, and the candidate structural variant 204n is in phase with a flanking variant 206e—each within respective contiguous sequences (or other nucleotide sequences) of the genomic sample database 202. Accordingly, as indicated by the dotted-line circles of FIG. 2A, in some embodiments, the structural-variant-aware sequencing system 106 selects the candidate structural variants 204c, 204d, 204g, and 204n as structural variant haplotypes to include within the structural variation graph genome 212.

In addition to selecting the candidate structural variants 204c, 204d, 204g, and 204n as structural variant haplotypes, as further shown in FIG. 2A, the structural-variant-aware sequencing system 106 identifies, from a linear reference genome 208, reference haplotypes 210a-210n corresponding to the selected structural variant haplotypes. For example, in some cases, the structural-variant-aware sequencing system 106 identifies the reference haplotypes 210a-210n at genomic coordinates of the linear reference genome 208 corresponding to the selected structural variant haplotypes. Indeed, the structural-variant-aware sequencing system 106 can identify genomic coordinates of the reference haplotypes 210a-210n above which to incorporate the selected variant haplotypes as liftover groups in the structural variation graph genome 212.

As further shown in FIG. 2A, the structural-variant-aware sequencing system 106 generates the structural variation graph genome 212. As shown, the structural variation graph genome 212 comprises alternate contiguous sequences 214a, 214b, 214c, and 214n representing the selected structural variant haplotypes. In some embodiments, one or more of the alternate contiguous sequences also include flanking variants 206a-206e.

To organize different structural variant haplotypes for a particular genomic region, in certain cases, the structural-variant-aware sequencing system 106 generates the structural variation graph genome 212 by ordering different subsets of alternate contiguous sequences corresponding to different genomic regions according to structural variant frequency within the genomic sample database 202. Accordingly, in some cases, the structural-variant-aware sequencing system 106 generates the structural variation graph genome 212 by ordering (i) a first subset of alternate contiguous sequences corresponding to a first genomic region according to frequency within the genomic sample database 202 and (ii) a second subset of alternate contiguous sequences corresponding to a second genomic region according to frequency within the genomic sample database 202.

As further shown in FIG. 2A, the structural variation graph genome 212 comprises reference sequences 216a, 216b, 216c, and 216n representing the reference haplotypes corresponding to the selected structural variant haplotypes. Indeed, in some embodiments, the structural variation graph genome 212 includes and is backwards compatible with the linear reference genome 208. As explained further below, in some embodiments, the structural-variant-aware sequencing system 106 generates the structural variation graph genome 212 by constructing a hash table or other organizational structure.

In addition or in the alternative to generating the structural variation graph genome 212, in some embodiments, the structural-variant-aware sequencing system 106 aligns nucleotide reads of a genomic sample with the structural variation graph genome 212 and determines nucleobase calls for the genomic sample based on the aligned nucleotide reads. FIG. 2B depicts an example of one such implementation of the structural variation graph genome 212. As shown in FIG. 2B, the structural-variant-aware sequencing system 106 identifies or receives nucleotide reads 218 for a genomic sample. In some cases, for instance, the structural-variant-aware sequencing system 106 receives base-call data (e.g., BCL file or FASTQ file) from a sequencing device, which has sequenced oligonucleotides extracted from the genomic sample and determined individual nucleobase calls for the nucleotide reads 218 in the base-call data. Depending on the type of sequencing performed, in some embodiments, the structural-variant-aware sequencing system 106 identifies either single-end reads or paired-end reads and either short nucleotide reads (e.g., <300 base pairs or <10,000 base pairs) or long nucleotide reads (e.g., >300 base pairs or >10,000 base pairs) as the nucleotide reads 218.

As further shown in FIG. 2B, the structural-variant-aware sequencing system 106 aligns the nucleotide reads 218 with different sequences of the structural variation graph genome 212. In particular, the structural-variant-aware sequencing system 106 aligns a subset of nucleotide reads 220 from the nucleotide reads 218 with the alternate contiguous sequence 214b of the structural variation graph genome 212. As FIG. 2B suggests, some or all of the subset of nucleotide reads 220 overlap with the alternate contiguous sequence 214b. In this particular example, the subset of nucleotide reads 220 overlap with the alternate contiguous sequence 214b representing the candidate structural variant 204f—that is, an insertion exceeding a threshold number of bases.

In addition to the alternate contiguous sequence 214b, in some embodiments, the structural-variant-aware sequencing system 106 aligns different subsets of nucleotide reads for the genomic sample with one or more of the alternate contiguous sequences 214a, 214c, or 214n or the reference sequences 216a-216n of the structural variation graph genome 212. Accordingly, in certain implementations, the structural-variant-aware sequencing system 106 aligns certain nucleotide reads with alternate contiguous sequences representing different types of structural variant haplotypes, including, but not limited to, insertions, deletions, duplications, inversions, translocations, or CNVs. Likewise, in some cases, the structural-variant-aware sequencing system 106 aligns certain nucleotide reads with reference sequences representing reference haplotypes from the linear reference genome.

As further shown in FIG. 2B, the structural-variant-aware sequencing system 106 determines nucleobase calls 222 for the genomic sample based on the subset of nucleotide reads 220 aligning with the alternate contiguous sequence 214b. For example, the structural-variant-aware sequencing system 106 generates one or more variant calls corresponding to a structural variant haplotype represented by the alternate contiguous sequence 214b. The structural-variant-aware sequencing system 106 determines such variant calls in part because an alignment of the subset of nucleotide reads 220 with the alternate contiguous sequence 214b exhibits better mapping metrics, base-call-quality metrics, or other sequencing metrics than an alignment of the subset of nucleotide reads 220 with the reference sequence 216b. In some embodiments, the structural-variant-aware sequencing system 106 generates a variant call file 224 comprising the nucleobase calls 222 along with other nucleobase calls based on read alignments.

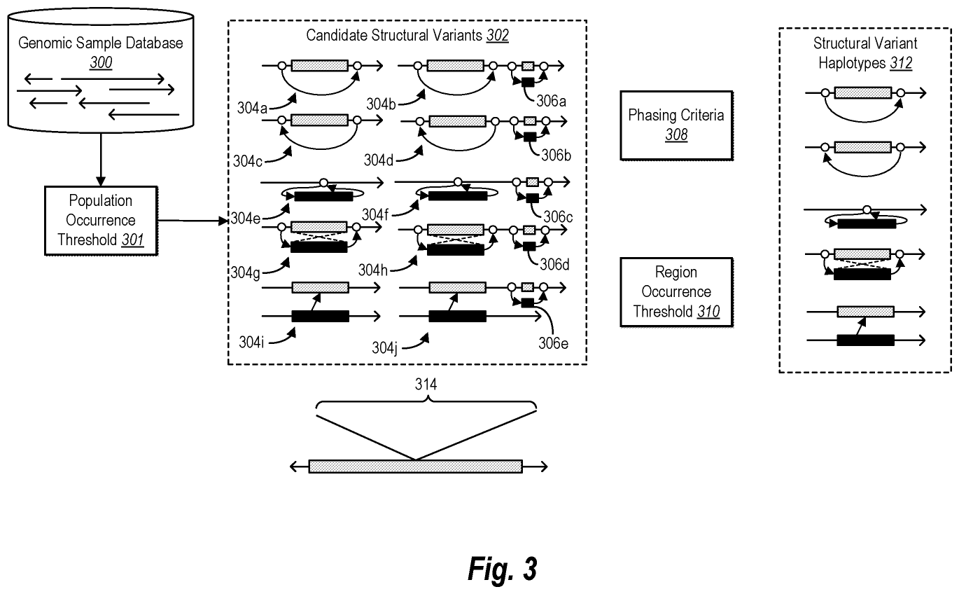

As noted above, the structural-variant-aware sequencing system 106 can select structural variant haplotypes from a genomic sample database to include within a structural variation graph genome. In accordance with one or more embodiments, FIG. 3 illustrates the structural-variant-aware sequencing system 106 selecting structural variant haplotypes for a target genomic region of a structural variation graph genome based on one or both of a phasing criteria 308 and a region occurrence threshold 310. Although not shown in FIG. 3, in certain implementations, the structural-variant-aware sequencing system 106 also selects structural variant haplotypes for other target genomic regions to include within a structural variation graph genome consistent with the disclosure below.

As suggested by FIG. 3, the structural-variant-aware sequencing system 106 identifies candidate structural variants from a genomic sample database 300. Consistent with the disclosure above, the genomic sample database 300 may include long nucleotide reads (e.g., reads >300 base pairs or >1,000 base pairs) that comprise various structural variants. In some embodiments, the genomic sample database 300 comprises contiguous sequences from a diverse set of genomic samples (e.g., from different geographical regions or countries of the world) that comprise structural variants. Indeed, the genomic sample database 300 may include contiguous sequences organized according to haplotype that comprise one or more structural variants. As explained below, the structural-variant-aware sequencing system 106 can leverage some such contiguous sequences that comprise flanking variants in phase with corresponding structural variant haplotypes for improved alignment, mapping, and base calling.

As depicted in FIG. 3, in some embodiments, the structural-variant-aware sequencing system 106 identifies the candidate structural variants based on a population occurrence threshold 301. The population occurrence threshold 301 provides an example of a threshold quantity of occurrences. For example, the structural-variant-aware sequencing system 106 identifies the candidate structural variants that occur at or above a threshold frequency within a population represented by the genomic sample database 300. In some cases, the threshold frequency constitutes a particular percentage (e.g., 1%, 5%) of genomic samples represented by contiguous sequences (or other nucleotide sequences) within the genomic sample database 300. Additionally or alternatively, the structural-variant-aware sequencing system 106 identifies the candidate structural variants that occur at or above a threshold count within genomic samples represented by contiguous sequences (or other nucleotide sequences) within the genomic sample database 300. In some cases, the threshold count constitutes a particular number (e.g., 3, 10, 25, 100) of genomic samples represented by such contiguous sequences or other nucleotide sequences within the genomic sample database 300.

In addition to generally identifying candidate structural variants from a population, in some embodiments, the structural-variant-aware sequencing system 106 determines candidate structural variants corresponding to particular genomic regions. As shown in FIG. 3, for instance, the structural-variant-aware sequencing system 106 identifies candidate structural variants 302 for a target genomic region 314. In some cases, the target genomic region 314 represents a gene, promoter region, or other genomic region.

For a given target genomic region, the structural-variant-aware sequencing system 106 may identify different types of candidate structural variants. As shown by FIG. 3, for example, the structural-variant-aware sequencing system 106 identifies candidate structural variants 302 for the target genomic region 314. Based on satisfying the population occurrence threshold 301 the structural-variant-aware sequencing system 106 identifies, for the target genomic region 314, the candidate structural variants 304a and 304b exhibiting deletions exceeding a threshold number of base pairs (e.g., <50, 100, or 1,000 base pairs); the candidate structural variants 304c and 304d exhibiting duplications exceeding a threshold number of base pairs; the candidate structural variants 304e and 304f exhibiting insertions exceeding a threshold number of base pairs; the candidate structural variants 304g and 304h exhibiting inversions; and the candidate structural variants 304i and 304j exhibiting translocations.

For illustrative purposes and space constraints, FIG. 3 depicts the candidate structural variants 204a-204n as merely examples for the target genomic region 314. The structural-variant-aware sequencing system 106 may identify, from the genomic sample database 300, different types of structural variants (e.g., CNVs) and fewer or more structural variants than depicted in FIG. 3 for the target genomic region 314. Likewise, in some embodiments, the structural-variant-aware sequencing system 106 may identify, from the genomic sample database 300, a different group of candidate structural variants (or no candidate structural variants) for different target genomic regions corresponding to genomic coordinates of a linear reference genome.

In addition to identifying the candidate structural variants 302, in some embodiments, the structural-variant-aware sequencing system 106 selects structural variant haplotypes 312 from among the candidate structural variants 302 based on one or both of the phasing criteria 308 and the region occurrence threshold 310. For example, in certain implementations, the structural-variant-aware sequencing system 106 selects the structural variant haplotypes 312 based on the phasing criteria 308 by selecting structural variant haplotypes that are respectively in phase with flanking variants within contiguous sequences. As shown in FIG. 3, the candidate structural variants 304b, 304d, 304f, 304h, and 304j are in phase with flanking variants 306a, 306b, 306c, 306d, and 306e, respectively, that are adjacent to the candidate structural variants 304b, 304d, 304f, 304h, and 304j within respective contiguous sequences. By contrast, the candidate structural variants 304a, 304e, 304e, 304g, and 304i are not in phase with a flanking variant within respective contiguous sequences. Accordingly, in some embodiments, the structural-variant-aware sequencing system 106 selects the candidate structural variants 304b, 304d, 304f, 304h, and 304j as the structural variant haplotypes 312 for the target genomic region 314. In some such embodiments, the structural-variant-aware sequencing system 106 removes or filters out the candidate structural variants 304a, 304e, 304e, 304g, and 304i from consideration.

By selecting structural variant haplotypes that are respectively in phase with flanking variants within contiguous or other nucleotide sequences, the structural-variant-aware sequencing system 106 can select structural variant haplotypes that facilitate better mapping and alignment with nucleotide reads in a structural variation graph genome than other structural variant haplotypes that lack such phased flanking variants. When a structural variation graph genome includes structural variant haplotypes with such phased flanking variants, the structural-variant-aware sequencing system 106 is more likely to align nucleotide reads of a genomic sample comprising some or all of a corresponding structural variant when the nucleotide reads likewise include a flanking variant also represented by an alternate contiguous sequence of the structural variation graph genome. When mapped to an alternate contiguous sequence with a flanking variant of the structural variation graph genome, the structural-variant-aware sequencing system 106 is also more likely to determine a relatively higher mapping-quality metric (e.g., MAPQ) and local alignment score (e.g., Smith-Waterman score) of mapping and alignment of a nucleotide read to the alternate contiguous sequence than to a reference sequence (or other alternate contiguous sequence) lacking such a flanking variant.

In addition or in the alternative to the phasing criteria 308, the structural-variant-aware sequencing system 106 selects the structural variant haplotypes 312 from among the candidate structural variants 302 based on the region occurrence threshold 310. The region occurrence threshold 310 provides another example of a threshold quantity of occurrences. For example, the structural-variant-aware sequencing system 106 selects the structural variant haplotypes 312 by selecting candidate structural variants that occur at or above a threshold frequency at the target genomic region 314. In some cases, the threshold frequency constitutes a particular percentage (e.g., 10%, 25%) of genomic samples represented by contiguous sequences (or other nucleotide sequences) within the genomic sample database 300 for the target genomic region 314 (e.g., at least one overlapping genomic coordinate with the target genomic region 314). Additionally or alternatively, the structural-variant-aware sequencing system 106 selects the structural variant haplotypes 312 by selecting candidate structural variants that occur at or above a threshold count within contiguous sequences (or other nucleotide sequences) within the genomic sample database 300 for the target genomic region 314 (e.g., at least one overlapping genomic coordinate). In some cases, the threshold count constitutes a particular number (e.g., 3, 10, 15) of contiguous sequences or other nucleotide sequences corresponding to the target genomic region 314.

By selecting structural variant haplotypes based on one or both of the phasing criteria 308 and the region occurrence threshold 310, in some cases, the structural-variant-aware sequencing system 106 improves the computing speed and memory of sequencing systems using certain graph reference genomes. In contrast to a generic graph reference genome that would include alternate contiguous sequences for largely irrelevant or excessive alleles at target genomic regions, the structural-variant-aware sequencing system 106 reduces the memory required to save a relatively smaller structural variation graph genome in terms of more targeted alternate contiguous sequences and corresponding structural variant haplotypes. Rather than an indiscriminate number of alternate contiguous sequences in a generic graph reference genome, in some embodiments, the structural-variant-aware sequencing system 106 intelligently selects targeted alternate contiguous sequences representing structural variant haplotypes based on one or both of the phasing criteria 308 and the region occurrence threshold 310.

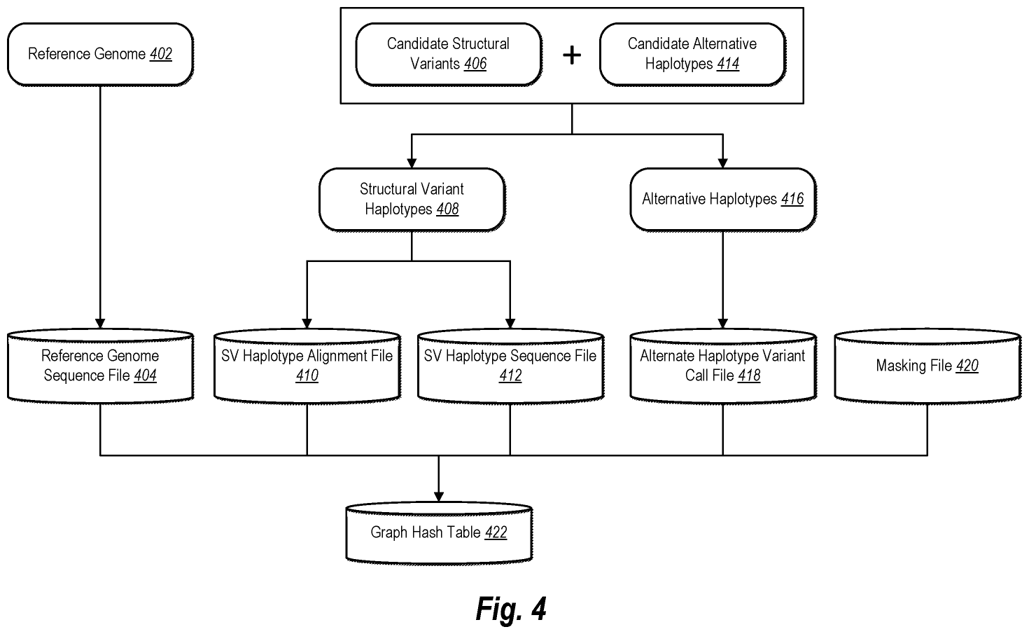

After selecting structural variant haplotypes and other alternate haplotypes, the structural-variant-aware sequencing system 106 can generate a structural variation graph genome using a digital organizational structure. In accordance with one or more embodiments, FIG. 4 illustrates the structural-variant-aware sequencing system 106 combining reference sequences from a reference genome, selected structural variant haplotypes, and selected alternate haplotypes into a structural variation graph genome using a graph hash table. As explained below, the graph hash table associates encoded nucleotide sequences for reference sequences, selected structural variant haplotypes, and selected alternate haplotypes with genomic coordinates.

In addition to selecting structural variant haplotypes as depicted in FIG. 2B or 3, in some embodiments, the structural-variant-aware sequencing system 106 identifies or selects alternate haplotypes for inclusion within a structural variation graph genome. For instance, in some cases, the structural-variant-aware sequencing system 106 selects one or more of SNPs, deletions of less than a threshold number of base pairs (e.g., >50 base pairs), or insertions of less than the threshold number of base pairs from a genomic sample database. Such alternate haplotypes differ in size and (in some cases) kind from structural variant haplotypes. Consistent with the disclosure above, in some such cases, the structural-variant-aware sequencing system 106 selects alternate haplotypes based on a region occurrence threshold for target genomic regions of a linear reference genome. Having selected alternate haplotypes, in some embodiments, the structural-variant-aware sequencing system 106 generates a structural variation graph genome comprising (i) reference sequences representing reference haplotypes, (ii) alternate contiguous sequences representing selected structural variant haplotypes, and (iii) alternate nucleobases or additional alternate contiguous sequences representing selected alternate haplotypes.

To organize and relate such reference sequences, alternate nucleobases, and alternate contiguous sequences, in some embodiments, the structural-variant-aware sequencing system 106 generates a digital organizational structure that associates the aforementioned reference and alternate sequences with genomic coordinates. For example, in certain implementations, the structural-variant-aware sequencing system 106 generates an alignment file that maps the selected structural variant haplotypes to genomic coordinates of the selected reference haplotypes within a linear reference genome. In some cases, the alignment file constitutes a Sequence Alignment/Map (SAM) liftover file. By leveraging the alignment file, the structural-variant-aware sequencing system 106 generates the structural variation graph genome by associating, within an organization structure (e.g., a hash table), identifiers (e.g., single-letter codes, binary code) for the alternate contiguous sequences representing the structural variant haplotypes with values for the genomic coordinates of the reference haplotypes.

To integrate the reference sequences representing reference haplotypes and alternate nucleobases or additional alternate contiguous sequences, in some embodiments, the structural-variant-aware sequencing system 106 further generates files to represent the nucleobase or nucleotide sequences of reference haplotypes and selected alternate haplotypes. For instance, the structural-variant-aware sequencing system 106 generates a sequence file representing a reference genome comprising the reference haplotypes and a variant call file representing the selected alternate haplotypes. By leveraging the sequence file, the alignment file, and the variant call file, in some embodiments, the structural-variant-aware sequencing system 106 generates the structural variation graph genome by associating, within a hash table, nucleobase identifiers for (i) reference sequences representing reference haplotypes, (ii) alternate contiguous sequences representing selected structural variant haplotypes, and (iii) alternate nucleobases or additional alternate contiguous sequences with values representing the genomic coordinates of the reference haplotypes.

FIG. 4 illustrates the structural-variant-aware sequencing system 106 generating a graph hash table 422 as such an organizational structure based on corresponding files. As shown in FIG. 4, for instance, the structural-variant-aware sequencing system 106 identifies a reference genome 402, such as a linear reference genome. For example, the structural-variant-aware sequencing system 106 identifies GRCh38 (or other versions of reference genomes) from the Genome Reference Consortium as the reference genome 402. Based on the reference genome 402, the structural-variant-aware sequencing system 106 generates a reference genome sequence file 404 comprising an encoded version of the reference genome 402. For instance, in some embodiments, the structural-variant-aware sequencing system 106 generates a FASTA format file as the reference genome sequence file 404. Such a FASTA file comprises text with single-letter codes (e.g., A, C, T, G, U, R, Y, M, S, W) representing nucleobases (e.g., A, C, T, G) of the nucleotide sequence of the reference genome 402.

In addition to the reference genome 402, as further shown in FIG. 4, the structural-variant-aware sequencing system 106 identifies candidate structural variants 406 from a genomic sample database and selects structural variant haplotypes 408 from among the candidate structural variants 406 for inclusion in a structural variation graph genome. For instance, the structural-variant-aware sequencing system 106 selects the structural variant haplotypes 408 using the method illustrated by FIG. 3 and described above. Accordingly, in some cases, the structural variant haplotypes 408 comprise structural variant haplotypes that are in phase with flanking variants (e.g., SNPs or indels) within contiguous sequences.

Based on the structural variant haplotypes 408, the structural-variant-aware sequencing system 106 generates a structural variant (SV) haplotype alignment file 410. For instance, the structural-variant-aware sequencing system 106 generates a Sequence Alignment/Map (SAM) liftover file that maps the structural variant haplotypes 408 to genomic coordinates of corresponding reference haplotypes within the reference genome 402. By generating a SAM liftover file, the structural-variant-aware sequencing system 106 generates a file that maps the structural variant haplotypes 408 to genomic coordinates for which alternate contiguous sequences will form liftover groups in a structural variation graph genome. Alternatively, the structural-variant-aware sequencing system 106 generates a Binary Alignment Map (BAM) file that compresses into a binary format such a mapping of the structural variant haplotypes to genomic coordinates of corresponding reference haplotypes.

Based on the structural variant haplotypes 408, as further shown in FIG. 4, the structural-variant-aware sequencing system 106 generates a structural variant (SV) haplotype sequence file 412. For instance, in some embodiments, the structural-variant-aware sequencing system 106 generates a FASTA format file as the SV haplotype sequence file 412. Such a FASTA file comprises text with single-letter codes representing individual nucleobases of the nucleotide sequence of the structural variant haplotypes 408. In some cases, the FASTA file includes descriptors or other headers identifying a target genomic region for individual structural variant haplotypes.

As further shown in FIG. 4, the structural-variant-aware sequencing system 106 identifies candidate alternate haplotypes 414. For instance, in some cases, the structural-variant-aware sequencing system 106 selects SNPs or indels below a threshold number of base pairs in low-confidence-call regions of the reference genome 402. To illustrate, a low-confidence-call region can include a genomic region including (in whole or in part) a variable number tandem repeat (VNTR), an insertion or deletion, or a region with a variety of different variations. A low-confidence-call region may likewise include genomic regions that have historically resulted in nucleobase calls that exhibit low-quality sequencing metrics, such as below a threshold base-call-quality metric (e.g., Q20, Q30, Q37) or a threshold mapping quality metric (e.g., a relative MAPQ score or MAPQ 40). Consistent with the disclosure above, in some embodiments, the structural-variant-aware sequencing system 106 selects the alternate haplotypes 416 based on a region occurrence threshold for target genomic regions of the reference genome 402, such as low-confidence-call regions.

Based on the alternate haplotypes 416, as further shown in FIG. 4, the structural-variant-aware sequencing system 106 generates an alternate haplotype variant call file 418. For example, the structural-variant-aware sequencing system 106 generates a VCF formatted file that identifies the alternate haplotypes with single-letter codes (e.g., A, T, C, G) to contrast with the single-letter codes for a corresponding reference haplotype at a particular genomic coordinate. In some embodiments, the structural-variant-aware sequencing system 106 generates a VCF file comprising more than 400,000 such alternate haplotypes for low-confidence-call regions.

Based on one or more of the reference genome sequence file 404, the SV haplotype alignment file 410, the SV haplotype sequence file 412, or the alternate haplotype variant call file 418, the structural-variant-aware sequencing system 106 generates the graph hash table 422. The graph hash table 422 represents an embodiment of a structural variation graph genome. For example, the structural-variant-aware sequencing system 106 generates the graph hash table 422 by associating each of (i) reference sequences representing reference haplotypes from the reference genome sequence file 404, (ii) alternate contiguous sequences representing the structural variant haplotypes 408 from the SV haplotype sequence file 412, and (iii) alternate nucleobases or additional alternate contiguous sequences from the alternate haplotype variant call file 418 with genomic coordinates of the reference haplotypes. The structural-variant-aware sequencing system 106 uses the SV haplotype alignment file 410 to map the structural variant haplotypes 408 to genomic coordinates over which alternate contiguous sequences will form liftover groups in the graph hash table 422. The graph hash table 422 accordingly represents an organizational structure that maps nucleobase identifiers (e.g., single-letter codes) of (i) reference haplotypes from the reference genome 402, (ii) the structural variant haplotypes 408, and (iii) the alternate haplotypes 416 to particular genomic coordinates.

In addition to the aforementioned files, in some embodiments, the structural-variant-aware sequencing system 106 generates a masking file 420. The masking file 420 partially masks the sequence or nucleobase identifiers (e.g., A, T, C, G) of the structural variant haplotypes 408 or the alternate haplotypes 416 with “N's” from as FASTA file. By masking the sequence or nucleobases of either or both of the structural variant haplotypes 408 or the alternate haplotypes 416, the structural-variant-aware sequencing system 106 can create a masked genome file based on custom annotations or mask (e.g., hide) target genomic regions when aligning sequence data from nucleotide reads. By using the masking file 420 to partially mask certain sequences, such as repeat sequences or low complexity genomic regions, the structural-variant-aware sequencing system 106 can selectively hide or mask reference sequences or alternative contiguous sequences for alignment—thereby ensuring that nucleotide reads are not aligned with such hidden nucleotide sequences. In some cases, the structural-variant-aware sequencing system 106 generates a browser extensible data (BED) file as the masking file 420. Accordingly, in some embodiments, certain nucleotide sequences in the graph hash table 422 are masked.

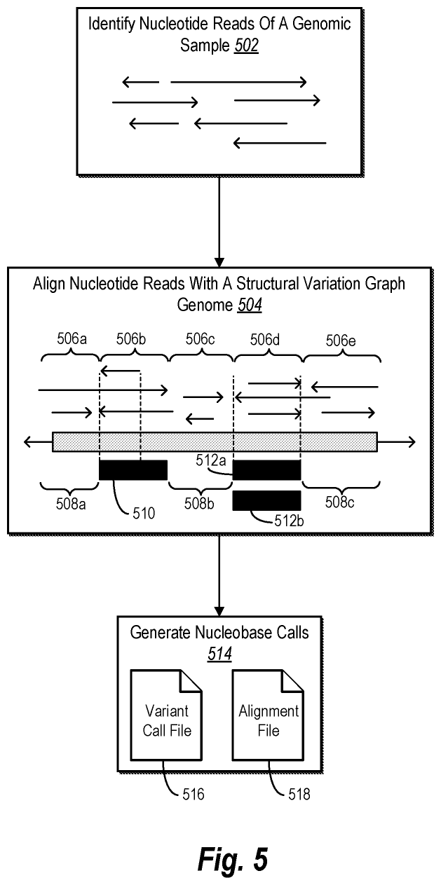

In addition or in the alternative to generating a structural variation graph genome in an organizational structure, in some embodiments, the structural-variant-aware sequencing system 106 implements the structural variation graph genome to determine variant calls or other nucleobase calls for genomic samples. In accordance with one or more embodiments, FIG. 5 illustrates the structural-variant-aware sequencing system 106 (i) aligning nucleotide reads of a genomic sample with a structural variation graph genome and (ii) determining nucleobase calls for the genomic sample based on the aligned nucleotide reads. As described below, the structural-variant-aware sequencing system 106 can determine variant calls (or other nucleobase calls) based on aligning a subset of nucleotide reads with alternate contiguous sequences representing structural variant haplotypes or alternate haplotypes.

As shown in FIG. 5, the structural-variant-aware sequencing system 106 identifies or receives nucleotide reads 502 for a genomic sample. In some cases, for instance, the structural-variant-aware sequencing system 106 receives base-call data (e.g., BCL file or FASTQ file) from a sequencing device. In some such cases, the base-call data takes the form of a base-call-data file that organizes single-end reads or paired-end reads according to index sequences attached to oligonucleotides extracted from a genomic sample. As indicated above, the structural-variant-aware sequencing system 106 can sequence or analyze short nucleotide reads (e.g., <300 base pairs or <10,000 base pairs) as the nucleotide reads 502, in some implementations, or long nucleotide reads (e.g., >300 base pairs or >10,000 base pairs) as the nucleotide reads 502, in other implementations.

As further shown in FIG. 5, the structural-variant-aware sequencing system 106 aligns the nucleotide reads 502 with different sequences within a structural variation graph genome 504. For instance, the structural-variant-aware sequencing system 106 aligns subsets of nucleotide reads 506a, 506c, and 506e in whole or in part with reference sequences 508a, 508b, and 508c, respectively. As indicated above, each of the reference sequences 508a-508c represent a different reference haplotype from a reference genome (e.g., GRCh38). As a further example, the structural-variant-aware sequencing system 106 aligns a subset of nucleotide reads 506b in whole or in part with an alternate nucleobase or an alternate contiguous sequence 510 representing an alternate haplotype. Finally, the structural-variant-aware sequencing system 106 aligns a subset of nucleotide reads 506d in whole or in part with an alternate contiguous sequence 512a (or an alternate contiguous sequence 512b) representing a structural variant haplotype.

For illustrative purposes and space constraints, FIG. 5 depicts the subsets of nucleotide reads 506a-506e, the reference sequences 508a-508c, the alternate nucleobase or the alternate contiguous sequence 510, and the alternate contiguous sequences 512a and 512b as merely examples. As indicated above, a sequencing device may generate numerous additional subsets of nucleotide reads, and the structural variation graph genome 504 may include numerous other types of reference sequences, alternate nucleobases, or alternate contiguous sequences. Indeed, the structural variation graph genome 504 depicted in FIG. 5 is merely one illustration to visualize reference sequences and alternate contiguous sequences of a structural variation graph genome embodied by a hash table, matrix, or other digital organizational structure.

As shown in FIG. 5, the structural-variant-aware sequencing system 106 determines that the subset of nucleotide reads 506d overlaps in whole or in part with the alternate contiguous sequence 512a representing a structural variant haplotype. For example, the structural-variant-aware sequencing system 106 determines that an alignment score (e.g., Smith-Waterman score or modified version of a Smith-Waterman score) exceeds other alignment scores for alternative alignments of the subset of nucleotide reads 506a with a corresponding reference sequence. Based in part on the alignment score for an alignment with the alternate contiguous sequence 512a exceeding other alignment scores for the subset of nucleotide reads 506d, in some embodiments, the structural-variant-aware sequencing system 106 generates a variant call indicating the genomic sample exhibits the structural variant haplotype represented by the alternate contiguous sequence 512a.

As just indicated, in some embodiments, the structural-variant-aware sequencing system 106 determines an alt-contig fragment alignment score (e.g., Smith-Waterman score or modified version of a Smith-Waterman score) for an alignment of the subset of nucleotide reads 506d with the alternate contiguous sequence 512a. The structural-variant-aware sequencing system 106 can also determine a split group score for a split alignment of the subset of nucleotide reads 506d with one or more reference sequences. If the alt-contig fragment alignment score exceeds the split group score for the split alignment of the subset of nucleotide reads 506d—and exceeds alignment scores (e.g., Smith-Waterman score) for other alignments with other alternate contiguous sequences, such as the alternate contiguous sequence 512b—the structural-variant-aware sequencing system 106 selects and reports a split alignment with a primary assembly of a reference genome corresponding to the alternate contiguous sequence 512a by a liftover relationship. By selecting and reporting such a split alignment, the structural-variant-aware sequencing system 106 can use the reported split alignment to determine nucleobase calls based on the alignment of the subset of nucleotide reads 506d with the alternate contiguous sequence 512a. However, if the split group score for the split alignment of the subset of nucleotide reads 506d exceeds the alt-contig fragment alignment score, the structural-variant-aware sequencing system 106 determines nucleobase calls based on a different split alignment with one or more reference sequences of the reference genome that may not represent an alignment with the alternate contiguous sequence 512a. In some embodiments, the structural-variant-aware sequencing system 106 determines alt-contig fragment alignment scores and split group scores as described by Improving Split-Read Alignment by Intelligently Identifying and Scoring Candidate Split Groups, U.S. Patent Application No. 63/367,002 (filed Jun. 24, 2022), which is hereby incorporated by reference in its entirety.

Based on aligning the subsets of nucleotide reads 506a-506e with different sequences of the structural variation graph genome 504, as further shown in FIG. 5, the structural-variant-aware sequencing system 106 generates nucleobase calls 514. For example, in some embodiments, the structural-variant-aware sequencing system 106 determines nucleobase calls for the subsets of nucleotide reads 506a, 506c, and 506e based on the alignments of the subsets of nucleotide reads 506a, 506c, and 506e with the reference sequences 508a, 508b, and 508c, respectively. In some such cases, the nucleobase calls may indicate references bases (e.g., represented as 0s) in a variant call file 516. As a further example of the nucleobase calls 514, the structural-variant-aware sequencing system 106 determines one or more variant calls for the subset of nucleotide reads 506b based on the alignment between the subset of nucleotide reads 506b and the alternate nucleobase or the alternate contiguous sequence 510.

Unlike existing sequencing systems, the structural-variant-aware sequencing system 106 can also determine variant calls corresponding to structural variants based on a structural variation graph genome. Based on an alignment of the subset of nucleotide reads 506a and the alternate contiguous sequence 512a, for example, the structural-variant-aware sequencing system 106 generates one or more variant calls indicating the genomic sample exhibits the structural variant haplotype represented by the alternate contiguous sequence 512a. In some cases, the structural-variant-aware sequencing system 106 generates the variant call file 516 or an alignment file 518 comprising (i) an annotation indicating one or more variant calls or other nucleobase calls represents the structural variant haplotype and/or (ii) an annotation indicating an alignment reflecting the structural variant haplotype within the genomic sample. Consistent with the disclosure above, the variant call or nucleobase call can correspond to a structural variant haplotype comprising a deletion of more than a threshold number of base pairs, an insertion of more than the threshold number of base pairs, a duplication of more than the threshold number of base pairs, an inversion, a translocation, or a copy number variation (CNV).

By aligning the subsets of nucleotide reads 506a-506e with alternate contiguous sequences of the structural variation graph genome 504 representing structural variant haplotypes, the structural-variant-aware sequencing system 106 can recover nucleobase calls that otherwise would not have been reported in output files. For example, in some embodiments, the structural-variant-aware sequencing system 106 determines that an alignment score for the subset of nucleotide reads 506d does not satisfy a threshold alignment score for a candidate alignment between the subset of nucleotide reads 506a and a primary-assembly region of a linear reference genome within the structural variation graph genome 504.

To illustrate such recovering, alignment scores for candidate alignments of the subset of nucleotide reads 506a with various reference sequences may fall below a threshold alignment score. By contrast, an alt-contig fragment alignment score for an alignment of the subset of nucleotide reads 506d with the alternate contiguous sequence 512a may satisfy the threshold alignment score. Accordingly, in some embodiments, the structural-variant-aware sequencing system 106 generates the variant call file 516 or the alignment file 518 with one or more nucleobase calls for the genomic sample based on the aligned subset of nucleotide reads 506d with the alternate contiguous sequence 512a—but without nucleobase calls for the genomic sample based on candidate alignments of the subset of nucleotide reads 506d with various reference sequences that do not satisfy the threshold alignment score.

As indicated above, the structural-variant-aware sequencing system 106 can generate the variant call file 516 or the alignment file 518 comprising annotations indicating information about a structural variant haplotype detected in a genomic sample. For instance, in one or more embodiments, the structural-variant-aware sequencing system 106 generates the variant call file 516 or the alignment file 518 comprising one or more of (i) an annotation indicating a variant call or other nucleobase call corresponds to a structural variant haplotype, (ii) an annotation indicating a frequency of the structural variant haplotype (e.g., a frequency within a genomic sample database of the structural variant haplotype), (iii) an annotation indicating genomic coordinates for the structural variant haplotype correspond to the nucleobase calls, or (iv) an annotation indicating an alignment reflecting the structural variant haplotype within the genomic sample.

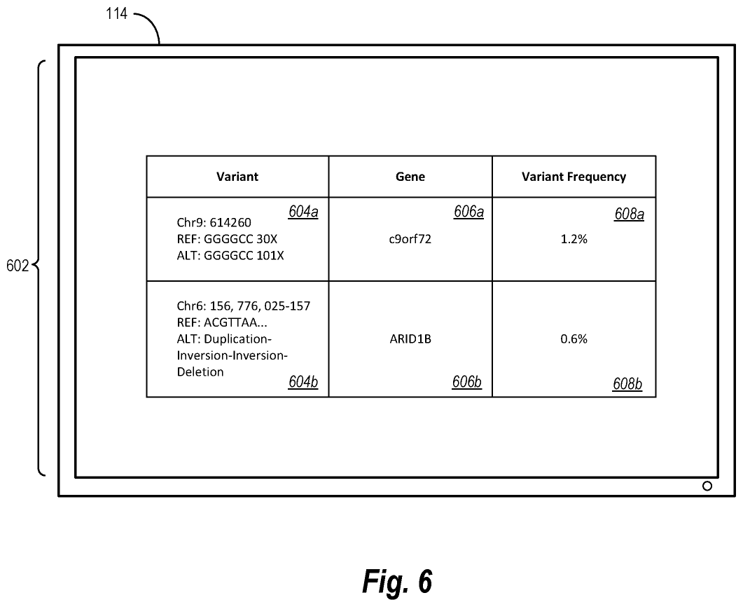

After generating data for one or more such annotations, in some embodiments, the structural-variant-aware sequencing system 106 provides the variant call file 516 or the alignment file 518 for display on a computing device. In accordance with one or more embodiments, FIG. 6 illustrates the client device 114 displaying a graphical user interface 602 comprising variant calls for structural variant haplotypes. While FIG. 6 depicts the graphical user interface 602 displayed when the client device 114 implements computer-executable instructions of the sequencing application 116, rather than repeatedly refer to the computer-executable instructions causing the client device 114 to perform certain actions for the structural-variant-aware sequencing system 106, this disclosure describes the client device 114 or the structural-variant-aware sequencing system 106 performing those actions in the following paragraphs. In some embodiments, the variant call file 516 or the alignment file 518 provide some of the computer-executable instructions and data to be presented within the graphical user interface 602.

As shown in FIG. 6, for instance, the client device 114 presents variant calls 604a and 604b reflecting different structural variant haplotypes exhibited by a genomic sample. Consistent with the disclosure above, the variant calls 604a and 604b represent graphical representations of nucleobase calls corresponding to structural variant haplotypes described above. As part of or in addition to each of the variant calls 604a and 604b, the client device 114 presents a reference-sequence indicator (e.g., REF: GGGGCC 30X or REF: ACGTTAA . . . ) for a reference sequence of a reference haplotype and an alternate-sequence indicator (e.g., ALT: GGGGCC 101X or ALT: Duplication-Inversion-Inversion-Deletion) for some or all of an alternate contiguous sequence corresponding to a structural variant haplotype. Further, as part of or in addition to each of the variant calls 604a and 604b, the client device 114 presents genomic coordinates for the variant calls 604a and 604b (e.g., Chr9: 614260 or Chr6: 156, 776, 025-157).

As further shown in FIG. 6, in some embodiments, the client device 114 presents annotations for a gene and a variant frequency corresponding to the variant calls 604a and 604b. For instance, the client device 114 presents genes 606a and 606b (e.g., c9orf72 or ARID1B) respectively corresponding to the variant calls 604a and 604b. In addition to specific gene identification, the client device 114 presents variant frequencies 608a and 608b (e.g., 1.2% or 0.6%) respectively indicating frequencies (e.g., from a genomic sample database) of the structural variant haplotypes represented by the variant calls 604a and 604b. By providing the reference-sequence identifiers, alternate-sequence identifiers, genomic coordinates, genes, and variant frequencies, the structural-variant-aware sequencing system 106 provides clinicians, test subjects, or other people with critical information indicating structural variant calls for certain genes.

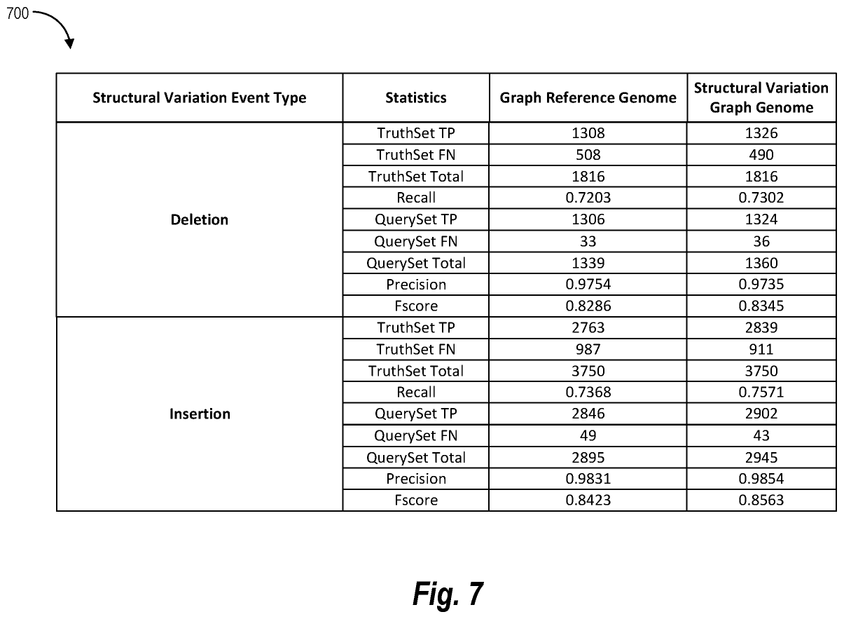

As indicated above, the structural-variant-aware sequencing system 106 improves the accuracy of read alignments and nucleobase calling by generating or utilizing a structural variation graph genome that represents structural variants. To test the accuracy of read alignments and nucleobase calling of the structural-variant-aware sequencing system 106, researchers compared the accuracy with which a sequencing system detects structural variants using an existing graph reference genome and the accuracy with which the structural-variant-aware sequencing system 106 identifies structural variants using a structural variation graph genome. In accordance with one or more embodiments, FIG. 7 illustrates a table 700 that shows different accuracy measurements of (i) a sequencing system determining variant calls for deletions and insertions exceeding 50 base pairs using an existing graph reference genome that lacks alternate contiguous sequences representing structural variants and (ii) the structural-variant-aware sequencing system 106 determining variant calls for such deletions and insertions using a structural variation graph genome. As the table 700 demonstrates, the structural-variant-aware sequencing system 106 improves true-positive genotype calls, false-negative genotype calls, recall rates, and F-scores of determining variant calls for deletions and insertions exceeding 50 base pairs by using a structural variation graph genome instead of an existing graph reference genome.

As indicated by FIG. 7, researchers input, into a sequencing system and the structural-variant-aware sequencing system 106, data for nucleotide reads from a query call set comprising new deletions and insertions exceeding 50 base pairs. The sequencing system aligned data for the nucleotide reads from the query call set with an existing graph reference genome, here the Illumina DRAGEN Graph Reference Genome hg19, and determined variant calls based on the aligned nucleotide read data. The structural-variant-aware sequencing system 106 also aligned data for the nucleotide reads in the query call set with an embodiment of a structural variation graph genome and determined variant calls based on the aligned nucleotide read data.

To evaluate the accuracy of genotype calls for the query call set, the researchers compared the genotype calls of the sequencing system and the structural-variant-aware sequencing system 106 for the query call set with a truth call set. The truth call set comprises known deletions and insertions exceeding 50 base pairs. For example, the truth call set includes a list of structural-variant events identified by either other technologies or manually validated.

As indicated by the table 700, the researchers further determined (i) a number of true positive (TP) genotype calls in which the sequencing system or the structural-variant-aware sequencing system 106 correctly determined corresponding insertions and deletions and (ii) a number of false negative (FN) genotype calls in which the sequencing system or the structural-variant-aware sequencing system 106 incorrectly determined no corresponding insertions and deletions. Based on the number of true positive and false negative genotype calls, the researchers also determined recall rates, precision rates, and F-score as indicated in the table 700.

As shown by the table 700, by using a structural variation graph genome instead of an existing graph reference genome, the structural-variant-aware sequencing system 106 improves the true positive genotype calls, reduces the false negative genotype calls, and improves the recall rate for deletions exceeding 50 base pairs in the truth call set. Similarly, by using the structural variation graph genome, the structural-variant-aware sequencing system 106 improves the true positive genotype calls, reduces the false negative genotype calls, improves the precision rate, and improves the F-score for deletions exceeding 50 base pairs in the query call set in comparison to the sequencing system's existing graph reference genome.

As further shown by the table 700, by using a structural variation graph genome instead of an existing graph reference genome, the structural-variant-aware sequencing system 106 improves the true positive genotype calls, reduces the false negative genotype calls, and improves the recall rate for insertions exceeding 50 base pairs in the truth call set. Similarly, by using the structural variation graph genome, the structural-variant-aware sequencing system 106 improves the true positive genotype calls, reduces the false negative genotype calls, improves the precision rate, and improves the F-score for insertions exceeding 50 base pairs in the query call set in comparison to the sequencing system's existing graph reference genome.

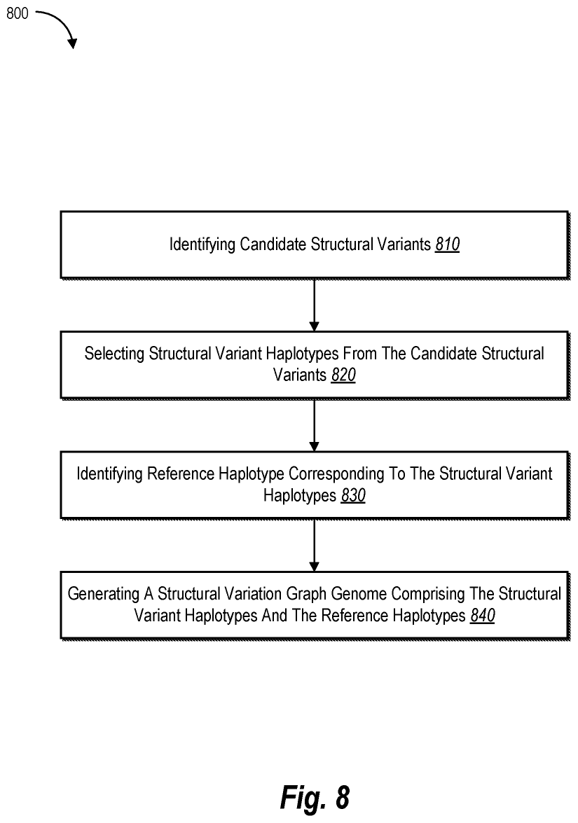

Turning now to FIG. 8, this figure illustrates a flowchart of a series of acts 800 of generating a structural variation graph genome in accordance with one or more embodiments of the present disclosure. While FIG. 8 illustrates acts according to one embodiment, alternative embodiments may omit, add to, reorder, and/or modify any of the acts shown in FIG. 8. The acts of FIG. 8 can be performed as part of a method. Alternatively, a non-transitory computer readable storage medium can comprise instructions that, when executed by one or more processors, cause a computing device or a system to perform the acts depicted in FIG. 8. In still further embodiments, a system comprising at least one processor and a non-transitory computer readable medium comprising instructions that, when executed by one or more processors, cause the system to perform the acts of FIG. 8. In some cases, the at least one processor comprises a configurable processor and executing the at least one processor comprises configuring the configurable processor.

As shown in FIG. 8, the acts 800 include an act 810 of identifying candidate structural variants. In particular, in some embodiments, the act 810 includes identifying candidate structural variants that satisfy a threshold quantity of occurrences within a genomic sample database.

For example, in some cases, identifying the candidate structural variants comprises selecting structural variants representing one or more of a deletion of more than fifty base pairs, an insertion of more than fifty base pairs, a duplication of more than fifty base pairs, an inversion, a translocation, or a copy number variation (CNV). As a further example, in certain cases, identifying the candidate structural variants comprises selecting structural variants representing one or more of a deletion of more than a threshold number of base pairs, an insertion of more than the threshold number of base pairs, a duplication of more than the threshold number of base pairs, an inversion, a translocation, or a copy number variation (CNV).

As further shown in FIG. 8, the acts 800 include an act 820 of selecting structural variant haplotypes from the candidate structural variants. In particular, in some embodiments, the act 820 includes selecting, from the candidate structural variants, structural variant haplotypes. For example, in some cases, selecting the structural variant haplotypes comprises selecting, from the candidate structural variants, particular structural variant haplotypes that satisfy an additional threshold quantity of occurrences at particular genomic regions.

To illustrate, in some embodiments, selecting the structural variant haplotypes comprises: selecting, from the candidate structural variants, a first structural variant haplotype that satisfies an additional threshold quantity of occurrences at a first genomic region; and selecting, from the candidate structural variants, a second structural variant haplotype that satisfies the additional threshold quantity of occurrences at a second genomic region.

Additionally, or alternatively, in some embodiments, selecting the structural variant haplotypes comprises selecting particular structural variant haplotypes adjacent to particular flanking variants within nucleotide sequences of the genomic sample database. In some cases, a flanking variant comprises a single nucleotide polymorphism (SNP), a deletion of less than fifty base pairs, or an insertion of less than fifty base pairs. In particular, in certain implementations, selecting the particular structural variant haplotypes comprises: selecting a first structural variant haplotype in phase with a first flanking variant within a first nucleotide sequence of the genomic sample database; and selecting a second structural variant haplotype in phase with a second flanking variant within a second nucleotide sequence of the genomic sample database.

To illustrate, in some embodiments, selecting the structural variant haplotypes comprises: selecting a first structural variant haplotype adjacent to a first flanking variant within a first nucleotide sequence of the genomic sample database; and selecting a second structural variant haplotype adjacent to a second flanking variant within a second nucleotide sequence of the genomic sample database. As indicated above, in some cases, the first flanking variant or the second flanking variant comprises a single nucleotide polymorphism (SNP), a deletion of less than a threshold number of base pairs, or an insertion of less than the threshold number of base pairs.

As further shown in FIG. 8, the acts 800 include an act 830 of identifying reference haplotype corresponding to the structural variant haplotypes. In particular, in certain implementations, the act 830 includes identifying, from a linear reference genome, reference haplotypes corresponding to the structural variant haplotypes.

As further shown in FIG. 8, the acts 800 include an act 840 of generating a structural variation graph genome comprising the structural variant haplotypes and the reference haplotypes. In particular, in certain implementations, the act 840 includes generating a structural variation graph genome comprising alternate contiguous sequences representing the structural variant haplotypes and reference sequences representing the reference haplotypes. As suggested above, in some embodiments, the act 840 includes generating the structural variation graph genome comprising particular alternate contiguous sequences representing the particular structural variant haplotypes and the particular flanking variants.

To illustrate, in some cases, generating the structural variation graph genome comprises generating the structural variation graph genome comprising: a first alternate contiguous sequence representing a first structural variant haplotype and a first flanking variant; and a second alternate contiguous sequence representing a second structural variant haplotype and a second flanking variant. Further, in some cases, generating the structural variation graph genome comprises ordering a subset of alternate contiguous sequences corresponding to a genomic region according to frequency within the genomic sample database.

In addition or in the alternative to the acts 810-840, in certain implementations, the acts 800 further include identifying, from the genomic sample database, alternate haplotypes comprising one or more of a single nucleotide polymorphism (SNP), a deletion of less than fifty base pairs, or an insertion of less than fifty base pairs; and generating the structural variation graph genome further comprising alternate nucleobases or additional alternate contiguous sequences representing the alternate haplotypes.

As suggested above, in some embodiments, the acts 800 include generating an alignment file that maps the structural variant haplotypes to genomic coordinates of the reference haplotypes within the linear reference genome; and generating the structural variation graph genome by associating, within an organization structure, the alternate contiguous sequences representing the structural variant haplotypes with identifiers for the genomic coordinates of the reference haplotypes. For instance, in certain implementations, generating the alignment file comprises generating a Sequence Alignment/Map (SAM) liftover file that maps the structural variant haplotypes to the genomic coordinates of the reference haplotypes; and generating the structural variation graph genome comprises generating the structural variation graph genome utilizing the organization structure by associating, within a hash table, nucleobase identifiers for nucleobases from the alternate contiguous sequences with values representing the genomic coordinates of the reference haplotypes.

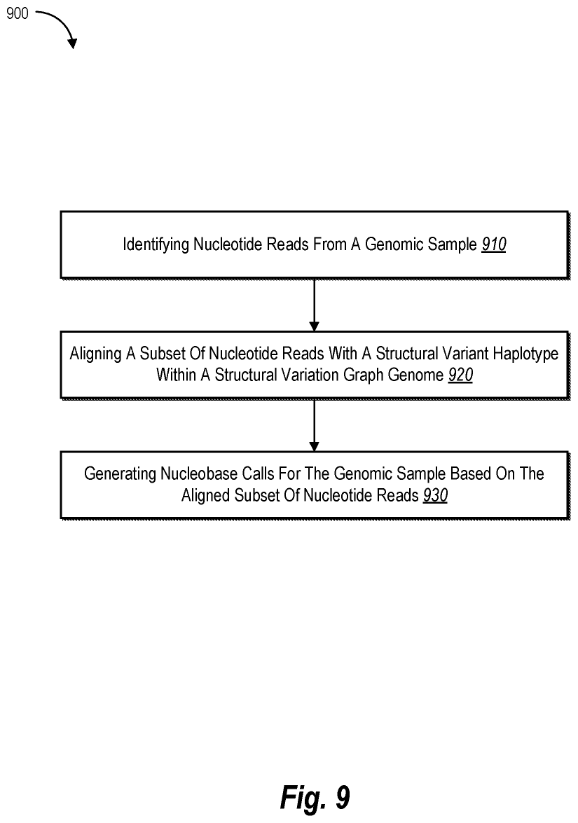

Turning now to FIG. 9, this figure illustrates a flowchart of a series of acts 900 of aligning nucleotide reads of a genomic sample with a structural variation graph genome and determining nucleobase calls for the genomic sample based on the aligned nucleotide reads in accordance with one or more embodiments of the present disclosure. While FIG. 9 illustrates acts according to one embodiment, alternative embodiments may omit, add to, reorder, and/or modify any of the acts shown in FIG. 9. The acts of FIG. 9 can be performed as part of a method. Alternatively, a non-transitory computer readable storage medium can comprise instructions that, when executed by one or more processors, cause a computing device or a system to perform the acts depicted in FIG. 9. In still further embodiments, a system comprising at least one processor and a non-transitory computer readable medium comprising instructions that, when executed by one or more processors, cause the system to perform the acts of FIG. 9. In some cases, the at least one processor comprises a configurable processor and executing the at least one processor comprises configuring the configurable processor.

As shown in FIG. 9, the acts 900 include an act 910 of identifying nucleotide reads from a genomic sample. As further shown in FIG. 9, the acts 900 include an act 920 of aligning a subset of nucleotide reads with a structural variant haplotype within a structural variation graph genome. In particular, in some embodiments, the act 920 includes aligning a subset of nucleotide reads with an alternate contiguous sequence representing a structural variant haplotype within a structural variation graph genome.

In some cases, the structural variant haplotype comprises a deletion of more than fifty base pairs, an insertion of more than fifty base pairs, a duplication, an inversion, a translocation, or a copy number variation (CNV). Alternatively, in certain cases, the structural variant haplotype comprises a deletion of more than a threshold number of base pairs, an insertion of more than the threshold number of base pairs, a duplication of more than the threshold number of base pairs, an inversion, a translocation, or a copy number variation (CNV).

As further shown in FIG. 9, the acts 900 include an act 930 of generating nucleobase calls for the genomic sample based on the aligned subset of nucleotide reads. In particular, in certain implementations, the act 930 includes generating one or more nucleobase calls for the genomic sample based on the aligned subset of nucleotide reads.

In addition or in the alternative to the acts 910-930, in some embodiments, the acts 900 include generating an alignment file or a variant call file comprising an annotation indicating the structural variant haplotype corresponding to the one or more nucleobase calls. Additionally or alternatively, in some cases, the acts 900 include generating an alignment file or a variant call file comprising an annotation indicating a frequency within a genomic sample database of the structural variant haplotype corresponding to the one or more nucleobase calls. Additionally or alternatively, in certain embodiments, the acts 900 include generating an alignment file or a variant call file comprising genomic coordinates of a linear reference genome that is part of the structural variation graph genome and that corresponds to the one or more nucleobase calls.

As suggested above, in some embodiments, the acts 900 include determining that the subset of nucleotide reads overlap with a breakpoint of the alternate contiguous sequence representing the structural variant haplotype; and generating an alignment file or a variant call file comprising an annotation indicating an alignment reflecting the structural variant haplotype within the genomic sample.

Additionally or alternatively, in certain implementations, the acts 900 include determining that an alignment score for the subset of nucleotide reads does not satisfy a threshold alignment score for a candidate alignment between the subset of nucleotide reads and a primary-assembly region of a linear reference genome; and generating a variant call file or an alignment file with the one or more nucleobase calls for the genomic sample based on the aligned subset of nucleotide reads with the alternate contiguous sequence and without nucleobase calls for the genomic sample based on the candidate alignment that does not satisfy the threshold alignment score.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the structural-variant-aware sequencing system 106 can include software, hardware, or both. For example, the components of the structural-variant-aware sequencing system 106 can include one or more instructions stored on a computer-readable storage medium and executable by processors of one or more computing devices (e.g., the client device 114). When executed by the one or more processors, the computer-executable instructions of the structural-variant-aware sequencing system 106 can cause the computing devices to perform the bubble detection methods described herein. Alternatively, the components of the structural-variant-aware sequencing system 106 can comprise hardware, such as special purpose processing devices to perform a certain function or group of functions. Additionally, or alternatively, the components of the structural-variant-aware sequencing system 106 can include a combination of computer-executable instructions and hardware.

Furthermore, the components of the structural-variant-aware sequencing system 106 performing the functions described herein with respect to the structural-variant-aware sequencing system 106 may, for example, be implemented as part of a stand-alone application, as a module of an application, as a plug-in for applications, as a library function or functions that may be called by other applications, and/or as a cloud-computing model. Thus, components of the structural-variant-aware sequencing system 106 may be implemented as part of a stand-alone application on a personal computing device or a mobile device. Additionally, or alternatively, the components of the structural-variant-aware sequencing system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the structural-variant-aware sequencing system 106 and the structural-variant-aware sequencing system 106. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
identify candidate structural variants that satisfy a threshold quantity of occurrences within a genomic sample database;
select, from the candidate structural variants, structural variant haplotypes;
identify, from a linear reference genome, reference haplotypes corresponding to the structural variant haplotypes; and
generate a structural variation graph genome comprising alternate contiguous sequences representing the structural variant haplotypes and reference sequences representing the reference haplotypes.

2. The system of claim 1, further comprising instructions that, when executed by the at least one processor, cause the system to select the structural variant haplotypes by:
selecting, from the candidate structural variants, a first structural variant haplotype that satisfies an additional threshold quantity of occurrences at a first genomic region; and
selecting, from the candidate structural variants, a second structural variant haplotype that satisfies the additional threshold quantity of occurrences at a second genomic region.

3. The system of claim 1, further comprising instructions that, when executed by the at least one processor, cause the system to select the structural variant haplotypes by:
selecting a first structural variant haplotype adjacent to a first flanking variant within a first nucleotide sequence of the genomic sample database; and
selecting a second structural variant haplotype adjacent to a second flanking variant within a second nucleotide sequence of the genomic sample database.

4. The system of claim 3, further comprising instructions that, when executed by the at least one processor, cause the system to identify the candidate structural variants by selecting structural variants representing one or more of a deletion of more than a threshold number of base pairs, an insertion of more than the threshold number of base pairs, a duplication of more than the threshold number of base pairs, an inversion, a translocation, or a copy number variation (CNV).

5. The system of claim 3, further comprising instructions that, when executed by the at least one processor, cause the system to generate the structural variation graph genome comprising:
a first alternate contiguous sequence representing a first structural variant haplotype and a first flanking variant; and
a second alternate contiguous sequence representing a second structural variant haplotype and a second flanking variant.

6. The system of claim 1, further comprising instructions that, when executed by the at least one processor, cause the system to:
generate an alignment file that maps the structural variant haplotypes to genomic coordinates of the reference haplotypes within the linear reference genome; and
generate the structural variation graph genome by associating, within an organization structure, the alternate contiguous sequences representing the structural variant haplotypes with identifiers for the genomic coordinates of the reference haplotypes.

7. The system of claim 6, further comprising instructions that, when executed by the at least one processor, cause the system to:
generate the alignment file by generating a Sequence Alignment/Map (SAM) liftover file that maps the structural variant haplotypes to the genomic coordinates of the reference haplotypes; and
generate the structural variation graph genome utilizing the organization structure by associating, within a hash table, nucleobase identifiers for nucleobases from the alternate contiguous sequences with values representing the genomic coordinates of the reference haplotypes.

8. The system of claim 1, further comprising instructions that, when executed by the at least one processor, cause the system to generate the structural variation graph genome by ordering a subset of alternate contiguous sequences corresponding to a genomic region according to frequency within the genomic sample database.

9. A non-transitory computer-readable medium comprising instructions that, when executed by at least one processor, cause a computing device to:
identify candidate structural variants that satisfy a threshold quantity of occurrences within a genomic sample database;
select, from the candidate structural variants, structural variant haplotypes;
identify, from a linear reference genome, reference haplotypes corresponding to the structural variant haplotypes; and
generate a structural variation graph genome comprising alternate contiguous sequences representing the structural variant haplotypes and reference sequences representing the reference haplotypes.

10. The non-transitory computer-readable medium of claim 9, further comprising instructions that, when executed by the at least one processor, cause the computing device to select the structural variant haplotypes by selecting, from the candidate structural variants, particular structural variant haplotypes that satisfy an additional threshold quantity of occurrences at particular genomic regions.

11. The non-transitory computer-readable medium of claim 9, further comprising instructions that, when executed by the at least one processor, cause the computing device to select the structural variant haplotypes by selecting particular structural variant haplotypes adjacent to particular flanking variants within nucleotide sequences of the genomic sample database.

12. The non-transitory computer-readable medium of claim 11, further comprising instructions that, when executed by the at least one processor, cause the computing device to select the particular structural variant haplotypes by:
selecting a first structural variant haplotype in phase with a first flanking variant within a first nucleotide sequence of the genomic sample database; and
selecting a second structural variant haplotype in phase with a second flanking variant within a second nucleotide sequence of the genomic sample database.

13. The non-transitory computer-readable medium of claim 11, further comprising instructions that, when executed by the at least one processor, cause the computing device to generate the structural variation graph genome comprising particular alternate contiguous sequences representing the particular structural variant haplotypes and the particular flanking variants.

14. The non-transitory computer-readable medium of claim 9, further comprising instructions that, when executed by the at least one processor, cause the computing device to:
identify, from the genomic sample database, alternate haplotypes comprising one or more of a single nucleotide polymorphism (SNP), a deletion of less than fifty base pairs, or an insertion of less than fifty base pairs; and
generate the structural variation graph genome further comprising alternate nucleobases or additional alternate contiguous sequences representing the alternate haplotypes.

15. The non-transitory computer-readable medium of claim 9, further comprising instructions that, when executed by the at least one processor, cause the computing device to identify the candidate structural variants by selecting structural variants representing one or more of a deletion of more than fifty base pairs, an insertion of more than fifty base pairs, a duplication of more than fifty base pairs, an inversion, a translocation, or a copy number variation (CNV).

16. The non-transitory computer-readable medium of claim 9, wherein the at least one processor comprises a configurable processor and executing the at least one processor comprises configuring the configurable processor.

17. A method comprising:
identifying nucleotide reads from a genomic sample;
aligning a subset of nucleotide reads with an alternate contiguous sequence representing a structural variant haplotype within a structural variation graph genome; and
generating one or more nucleobase calls for the genomic sample based on the aligned subset of nucleotide reads.

18. The method of claim 17, further comprising generating an alignment file or a variant call file comprising one or more of an annotation indicating the structural variant haplotype corresponding to the one or more nucleobase calls, an annotation indicating a frequency within a genomic sample database of the structural variant haplotype corresponding to the one or more nucleobase calls, or genomic coordinates of a linear reference genome that is part of the structural variation graph genome and that corresponds to the one or more nucleobase calls.

19. The method of claim 17, further comprising:
determining that the subset of nucleotide reads overlap with a breakpoint of the alternate contiguous sequence representing the structural variant haplotype; and
generating an alignment file or a variant call file comprising an annotation indicating an alignment reflecting the structural variant haplotype within the genomic sample.

20. The method of claim 17, further comprising:
determining that an alignment score for the subset of nucleotide reads does not satisfy a threshold alignment score for a candidate alignment between the subset of nucleotide reads and a primary-assembly region of a linear reference genome; and
generating a variant call file or an alignment file with the one or more nucleobase calls for the genomic sample based on the aligned subset of nucleotide reads with the alternate contiguous sequence and without nucleobase calls for the genomic sample based on the candidate alignment that does not satisfy the threshold alignment score.
